(19) United States
(12) Patent Application Publication
(10) Pub. No.: US 2008/0084000 A1
(43) Pub. Date: Apr. 10, 2008

(54) NEW TYPE OF PHASE SEPARATION OF MICELLAR COLLOID SOLUTIONS

(76) Inventor: TIBOR FORSTER, Budapest (HU)

Correspondence Address: HAHN & VOIGHT PLLC, 1012 14TH STREET, NW, SUITE 620, WASHINGTON, DC, 20005, US

(21) Appl. No.: 11/870,937

(22) Filed: Oct. 11, 2007

Related U.S. Application Data
(63) Continuation-in-part of application No. PCT/HU2006/000027, filed on Apr. 12, 2006.

(30) Foreign Application Priority Data
Apr. 12, 2005 (HU) P0500379

(51) Int. Cl. B01J 13/20; C07C 211/13; C07C 211/22; C07H 21/00; C07K 14/00

(52) U.S. Cl. 264/4.3; 530/350; 536/22.1; 564/509; 564/512

(57) ABSTRACT

The invention provides colloid systems, preferably capsules, dispersions and emulsions, assembled by micelles containing new-type anionic surfactant and small molecular cationic complexing agent. Furthermore, the invention provides a method for phase separation, preferably for coacervation, of polymolecular colloid systems composed of aqueous anionic surfactants and the associates of them be the use of non colloidal sized complexing agents (polymolecular complex coacervation), and the use of this method for separating biologically active substances and for producing capsules, dispersions and emulsions containing such substances. Additionally, the invention provides reagent kits for performing the method of the invention. The colloid systems and methods of the invention can be very widely used for isolating, stabilizing or removing different substances, such as proteins, lipids, stains or dyes, radioactive substances, for isolating nucleic acids and for encapsulating or emulgeating different substances, such as proteins, active ingredients, stains or dyes, etc.

This is a continuation-in-part of International Application PCT/HU2006/000027, filed Apr. 12, 2006, the entire specification of which is hereby incorporated herein by reference. Applicant claims priority to Hungarian application P0500379, filed Apr. 12, 2005, the entire specification of which is also hereby incorporated herein by reference.

The invention relates to colloid systems formed by micelles comprising an anionic surfactant and a small molecular cationic complexing agent, whereby, the said colloid systems are preferably capsules, dispersion or emulsions. Furthermore, the invention relates to a method for phase-separation of polymolecular aqueous colloid systems of anionic surfactants and associations thereof; and to the use of said method for separation of biologically active materials and for the preparation of capsules, dispersions and emulsions comprising such materials. The phase separation is preferably coacervation (polymolecular complex coacervation). The invention further relates to reagent kits for use in the method of the invention.

### BACKGROUND OF THE INVENTION

The term “coacervation” was introduced in 1929 by Bungenberg de Jong and Kruyt, for a process in which aqueous colloidal solutions separate, upon the alteration of the thermodynamic condition of states, into two liquid phases. This process differs from precipitation, which can be observed in colloidaly instable systems, where a solution separates into a solid phase and a liquid phase. Coacervation is defined as the separation of colloidal systems into two liquid phases, the one rich in the colloid component, is called the coacervate, and the other is called equilibrium solution [see (1) IUPAC), Compendium of Chemical Terminology]. Considering different phase separation mechanisms, coacervation was subdivided into simple and complex coacervation. Simple coacervation occurs when highly hydrophilic substances are added (i.e. salting out by electrolytes, desolvation by organic solvents). Conversely, complex coacervation is essentially driven by the interaction of two oppositely charged colloids. (1)

The earliest commercial application of coacervation was for the development of “carbonless” carbon copy paper in the late 1950s, the process of which was established in the U.S. Pat. No. 1,800,457 patent disclosing the production of oil-containing microcapsules. According to this patent, the wall of the microcapsules is produced by the complex coacervation of a colloid system obtained by combining positively charged gelatine and negatively charged acacia gum.

Since the interest in microcapsules is increasing, numerous methods based on complex coacervation of two kinds of oppositely charged polyelectrolites have been developed so far. Procedures for the application of different polyelectrolites of artificial and natural origin have been established, the most popular of which are still represented by the protein-polysaccharide systems. The reason for this is the fact that while the surface charge of proteins can be firmly adjusted by changing the pH, the charge of the colloid remains constant. By accurately adjusting the pH, the absolute value of the surface charge of the two oppositely charged colloids can be equalized, which results in a complex having no surface charge and being composed of the two kinds of colloids. The procedure forms the basis of coacervation. International publication No. WO 0005446 describes the application of a gelatin-alginate complex for producing microcapsules. Due to its excellent gelation property, gelatine is preferable, even if, being a material of animal origin, there is a tendency to replace it with other materials nowadays.

As it is obvious from the U.S. Pat. No. 6,488,870 patent, the steps of the process for the preparation of microcapsules based on complex coacervation (such as emulsification, initiation of coacervation and capsule shell formation, the gelatinization of the wall of capsule followed by hardening) are essentially identical with the original conception. The quality of the capsule wall greatly affects the usability of microcapsules, thus it is a permanent task to improve the mechanical features of the wall. At present the formation of crosslinks between the components of the capsule wall is usually based on the use of aldehides (such as formaldehyde, glutaraldehyde, etc.).

Although numerous methods have been established for the complex coacervation-based production of microcapsules, there are certain disadvantages that are characteristic for all the methods. The methods allow using only accurately selected polyelectrolite pairs. Coacervation takes place only in a very narrow range of pH and concentration. Several parameters of the production (such as temperature, mixing, ionic strength, dilution, etc.) should be regulated in a highly accurate way. The required chemical feature and the mechanical stability of the microcell walls can be achieved only by additional treatment. The production is rather time-consuming.

Since a solution separates into a phase rich in colloid and one which is poor in it, the complex coacervation is often classified as liquid-liquid separation or associative phase separation. Although complex coacervation makes colloidal separation possible, in practice it is mainly used to prepare microcapsules and its application for separation is primarily mentioned in theoretical studies.

Xiao, J. X. et al. (2) studied the distribution of proteins in a two-phase system composed of cationic and anionic surfactant mixtures. The distribution coefficients of these proteins are so much different that the proteins can be separated by methods based on complex coacervation. The disadvantage of this type of methods is, on one hand, that it is highly sensitive to the proportional molar ratio of the surfactant and the pH, and on the other hand, the fact that coacervation is established only in two very narrow concentration ranges of the surfactants.

Wang, Y. F. et al. (3) has already tried to separate proteins, based on the finding that their coacervation with a polyelectrolite depends on the pH. It is evident from their study that the efficacy of the separation is very low, since, besides the pH, the process is also affected by other factors, such as ionic strength, the molecular weight of the polymer, etc. Furthermore, the charge distribution of the proteins and the cationic polyelectrolite are topologically different: the charge of the polymer is evenly distributed, while it is uneven on the proteins, which causes poor charge compensation.

From soy protein and SDS, Lazko, J et al. (4) produced an insoluble complex constituting a wall surrounding the oil drops. Similarly to the conventional method, the steps of the procedure are emulgeation, complex coacervation and crosslinking with glutaraldehyde. The procedure works only with a certain pH value, it is sensitive to the ratio of the components, and additional treatment is necessary to create a microcapsule wall with acceptable stability.

Wang, Y (5) studied a strong cationic polymer-induced coacervation of mixed micelles composed of Triton X-100 and SDS. The fact that coacervation occurs only in the case of polyelectrolite-micelle complexes with a size bigger than 45 nm indicates bad efficiency of the macromolecular charge compensation. The decrease or increase of the polyelectrolite-surfactant ratio equally inhibits coacervation, and the system is sensitive to the size of the polymer.

U.S. Pat. No. 4,801,691 describes a procedure of using guanidinium chloride for removing excess SDS from protein solutions solubilized with SDS. The method is useful for removing excess SDS not bound to protein. The method is not a coacervation. Guanidinium chloride is added at ambient temperature to the protein solution containing the excess SDS, then a part of the SDS precipitates as guanidinium-dodecyl-sulphate and the protein, as a complex with SDS, remains in solution. The procedure, although it was not its original purpose anyway, is not applicable for the separation of proteins. The authors did not observe any micelles composed of SDS and guanidinium chloride.

WO 9600228 describes a method for the isolation of nucleic acids from biological samples. By adding excess amount of salt (the authors were using two kinds of salt) to the lysis buffer containing the surfactant, such as SDS, the majority of proteins precipitate and the nucleic acids remain in the solution. Proteins which cannot be salted out are recommended to be removed by phenol treatment. Finally, the nucleic acids are precipitated. The method is used only as pretreatment and it contaminates the system with salt. In the procedure, the effective removal of proteins can be provided only by conventional phenol treatment.

The object of the invention is to provide multi-purpose methods allowing separation, stabilization or removal of micelles or materials (in particular proteins, lipids, dyes, radioactive materials, drugs) forming, directly or indirectly, association complex with said micelles, by improving the method of complex coacervation. A further object of the invention is to prepare microcapsules and similar formations using the coacervates.

By studying aqueous colloid systems it was unexpectedly found that certain self-assembling colloid systems, formed by association complexes of anionic surfactants or micelles thereof and of compounds bound directly or indirectly thereto by secondary chemical bonds, are capable of a novel type of complex coacervation unknown heretofore. According to the invention free negative charges of a polymolecular colloid system formed by anionic surfactants or micelles thereof and by compounds associated thereto, are neutralized by non-colloidal size complexing agents of small molecular weight, instead of cationic colloid particles or polycations used in the traditional complex coacervation. Said non-colloidal complexing agents are cationic and comprise groups capable of forming not only ionic but other intermolecular interactions, as well. The original counter-ions are displaced by complex-forming molecules, thereby polymolecular colloid particles are assembled, said particles comprising weakly solvated complexing molecules, surfactants and associates. Due to weak interactions between them, the colloid particles adhere to each other, then coacervate, i.e. finally form a separate liquid phase, the so-called coacervate. By the use of this method, in the presence of an apolar solvent, even reverse micelles can be formed. Moreover, the present inventors recognized that instead of coacervates a solid phase comprising the typical micelles of the invention can be obtained, which is useful in several applications.

### BRIEF DESCRIPTION OF THE INVENTION

In an aspect the present invention relates to a condensed phase composition comprising a micellar colloid system, wherein micelles comprise, as structural components, an anionic surfactant and a small molecular cationic complexing agent the latter being capable of forming complexes with the anionic surfactant by ionic interaction and by at least a further secondary interaction.

Preferably, in the composition
the anionic surfactant is capable of forming a hydrogen-bond and carries a negatively charged hydrogen-acceptor group, and
the cationic complexing agent is a hydrogen donor compound capable of forming at least a hydrogen bond as a further secondary interaction.

According to a preferred embodiment of the present invention, the hydrogen acceptor group present in the anionic surfactant is a group containing oxygen, preferably phosphate, carbonate, sulphate or sulphonate group, and the hydrophobic moiety of the anionic surfactant is a substituted or unsubstituted alkyl, alkenyl, alkinyl, aryl or aralkyl group; highly preferably the anionic surfactant is a biological membrane forming molecule, or its derivative, or a molecule capable of being integrated into biological membranes, or the hydrophilic moiety of the anionic surfactant is a sulphate- or sulphonate group or substance comprising these groups.

Preferably, the hydrophobic moiety of the anionic surfactant is a substituted or an unsubstituted alkyl, alkenyl or alkinyl group comprising at least 9, 10, 11 or 12 carbon atoms and, preferably, of at most 30, 25, 20 or 18 carbon atoms. Preferably, if the hydrophobic moiety is an alkenyl group, it comprises at most 5, 4, 3, 2 or 1 double bonds. Preferably, if the hydrophobic moiety is an alkinyl group, it comprises at most 5, 4, 3, 2 or 1 triple bonds. The alkyl, alkenyl or alkinyl group can be of a linear or a branched chain group. If it is of a branched chain alkyl, alkenyl or alkinyl group, preferably the one or more side chains of the branched chain is/are separately and independently from each other a methyl, ethyl, propyl, isopropyl, butyl, isobutyl, terc-butyl or any unsaturated derivative thereof.

If the hydrophobic moiety is a substituted alkyl, alkenyl or alkinyl group, the substituent should not substantially alter the hydrophobic character of the hydrophobic moiety. Thus, if the substituent is a halogen or a pseudohalogen, e.g. an F, Cl, Br, I, CN or SCN group, the hydrophobic moiety comprises preferably no more than 5, 4, 3, 2 or 1 substituent.

The substituent can be an aryl or arylalkyl group wherein the aryl moiety comprises preferably no more than 14, 10 or 6 carbon atoms. The substituent can also be any known heterocycle of sufficiently hydrophobic character. In a preferred embodiment the substituent is a chromophore of hydrophobic character, e.g. a fluorescent group or a light absorber allowing detection.

According to a further preferred embodiment of the present invention, the cationic complexing agent is a substance comprising ammonium cation or guanidinium cation. Preferably, as a cationic complexing agent, the composition comprises at least one of the following: a detectable molecule; a functional group capable of forming a covalent bond, preferably a group capable of binding conjugates and/or molecules containing a group capable of forming crosslinks, a molecule conjugated with a moiety which can be biologically recognized (such as an epitope) or is capable of biological recognition (such as a receptor); molecules comprising multiple complexing groups, such as molecules containing more than one, either protonated or unprotonated amino groups. Molecules containing more than one complexing groups may be capable of simultaneously linking several micelles, or capable of forming a link or links between micelles and other molecules, e.g. nucleic acids.

According to a further preferred embodiment of the present invention, in the composition of the present invention, the micelles of the composition may comprise proteins, nucleic acids, apolar substances, such as lipids, colouring agents or dyes, test compounds, pharmacologically active molecules, cosmetics, synthetic polymers, incorporated into or bound to the micelles, e.g. by forming a complex with them.

The composition of the present invention may comprise a single homogeneous phase, which can be in solid form comprising micelle-crystals, or the phase may be in liquid form which is a coacervate.

The composition of the present invention may comprise more than one phase, and may be e.g. in emulsified or in dispersed form.

Our experience shows that the invention is useful for storing and stabilizing compounds enclosed in or bound to micelles, such as compounds forming complexes with the micelles.

Therefore, the composition, e.g. the coacervate or the precipitate of the invention may be enclosed with or without the equilibrium solution, into an appropriate container, and can be stored or transported therein.

According to a preferred embodiment, in a composition suitable for storing compounds enclosed in the micelles or bound thereto, such as compounds forming complexes with them

the composition is a coacervate or it is a solid composition containing micelle crystals, and

the mixture is closed off from its environment with a covering medium that preserves the composition in the coacervate or solid state.

Preferably, the covering medium is a gel or the covering medium is a solution covered with solid material.

The micelles of the composition may comprise further compounds, preferably any of the following: proteins, nucleic acids or apolar compounds, such as lipids, stains or dyes, medicaments, cosmetics or synthetic polymers enclosed in the micelles or bound to the micelles, e.g. in a complex formed with them.

In another aspect of the present invention, the invention relates to microcapsules or nanocapsules comprising the composition of the invention.

In another preferred embodiment of the present invention, the micelles form micelle-crystals in the micro- or nanocapsules of the invention.

In a further preferred embodiment of the invention, at least a part of the complexing molecules present in the micro- or nanocapsules are crosslinked.

In a further preferred embodiment of the invention, molecules of the anionic surfactant and of the complexing agent in the micro- or nanocapsules of the invention are biocompatible, for example:

the anionic surfactant is a biological membrane-component or negatively charged derivative thereof, or an amphipatic molecule, such as a phospholipid, capable of being integrated into biological membranes; or

the complexing agent is an amino acid or an amino acid derivative, an oligopeptide or an oligopeptide-derivative, or any other, ammonium- or guanidium-comprising compound present in a living organism or harmless to any living organism.

According to another aspect of the present invention, the invention relates to the use of
a polar, protic solvent or a solution prepared with a polar, protic solvent,
an anionic surfactant suitable for forming micelles, when exposed to the said solvent or solution,
a cationic complexing agent which is soluble in the solvent, and its molecules are smaller than colloidal size, and wherein said agent, along with ionic interaction, can form cationic complexes via a further secondary interaction,

for preparing self-assembling coacervates of micelles comprising the anionic surfactant and the cationic complexing agent.

Preferably, the invention relates to, among others, the said use for separating substances interacting, at different levels, with micelles comprising the anionic surfactant and the cationic complexing agent, as well as to the use according to the invention for encapsulating substances interacting with micelles comprising the anionic surfactant and the cationic complexing agent. For example, the invention is suitable for encapsulating stains or dyes or biologically active compounds, such as test compounds, active ingredients, medicaments or cosmetics, or e.g. proteins, peptides, nucleic acids, etc.

According to another aspect the invention relates to a method for the preparation of a colloid system comprising micelles formed by anionic surfactants and for phase-separation thereof to a micelle rich phase (a phase more concentrated in micelles) and to an equilibrium solution (i.e. a micelle-depleted phase), preferably a method for coacervation, wherein

as a micelle rich phase, a self-assembling colloid system is prepared at least from the following:

a) a polar, protic solvent or solution containing it,

b) at least one type of anionic surfactant capable of forming micelles when exposed the solvent or the solution,

c) at least one type of small molecular, cationic complexing agent, which is soluble in the solvent and which can form complexes with the surfactant,

wherein

the anionic surfactant is used in a concentration above the Critical Micelle Concentration at a pH level ensuring that the anionic surfactant is in a charged form,

the cationic complexing agents are added to a concentration suitable for neutralizing the charges in the micelles,

after the addition of the surfactant and the cationic complexing agents the system is optionally homogenised, and

by providing an appropriate temperature and, if desired, by adjusting or changing the pH, ionic strength and/or dielectric constant, the phase-separation, preferably coacervation, of the colloid system is initiated.

Preferably, in the method or use of the present invention
the polar, protic solvent is water,
the anionic surfactant is negatively charged, capable of forming hydrogen bond and carries a hydrogen acceptor group, and
the cationic complexing agent is an agent with hydrogen donor property, capable of forming at least one hydrogen bond as a secondary interaction.

According to a preferred embodiment of the present invention, the hydrogen acceptor group is an oxygen comprising group, preferably phosphate, carbonate, sulphate or sulphonate group. Preferably, the hydrophobic moiety of the anionic surfactant is a substituted or unsubstituted alkyl, alkenyl, alkinyl, aryl or aralkyl group, while the hydrophilic moiety is a sulphate or sulphonate group or a substance comprising any of these groups. Any anionic surfactant satisfying the requirements described herein can be used in the invention.

According to a preferred embodiment of the present invention, a substance comprising an ammonium cation or a guanidinium cation can be used as a cationic complexing agent in the invention.

Preferably, the small molecular cationic complexing agent is non-colloidal.

Preferably, the small molecular cationic complexing agent is capable of effecting phase separation, preferably coacervation, in a phase separation method according to the invention. Preferably, in the phase separation method as a micelle rich phase, a self-assembling colloid system is prepared at least from the following:

a) a polar, protic solvent or solution containing it,

b) at least one type of anionic surfactant capable of forming micelles when exposed the solvent or the solution, wherein said anionic surfactant carries a negatively charged hydrogen acceptor group and is capable of forming hydrogen bonds,

c) at least one type of small molecular, cationic complexing agent with hydrogen donor property, which is soluble in the solvent and which, along with ionic interaction, can form complexes with the surfactant via at least hydrogen bond,

wherein

the anionic surfactant is used in a concentration above Critical Micelle Concentration at a pH level ensuring that the anionic surfactant is in a charged form and preferably at a temperature above the Kraft point,

the cationic complexing agent is added to a concentration suitable for neutralizing the charges in the micelles,

after addition of the surfactant and the cationic complexing agent the system is optionally homogenised, and

phase-separation, preferably coacervation, of the colloid system is initiated by providing an appropriate temperature and, if desired, by adjusting or changing the pH, ionic strength and/or dielectric constant.

Preferred cationic complexing agents are, for example, compounds comprising the cation of general formula (I):
R,R′,R″NH⁺, (I)

wherein

R is a hydroxyl or an unsubstituted or substituted alkoxy, aryloxy, alkyl, alkenyl, alkinyl or aryl group or, optionally an alkoxy, aryloxy, alkyl, alkenyl, alkinyl or aryl group substituted with one or more amino or imino groups or with a group comprising one or more amino or imino groups, or R is a substituted or an unsubstituted amino group.

R′ and R″ are H or substituted or unsubstituted alkyl, alkenyl, alkinyl or aryl group or, optionally, alkyl, alkenyl, alkinyl or aryl group substituted with one or more amino or imino groups or with a group comprising one or more amino or imino groups, or R and R′ are substituted or unsubstituted amino group; and R′ and R″ are identical with or different from each other.

In a preferred embodiment, one or more, preferably one or two, more preferably only one of R, R′ and R″ is or are a substituted or an unsubstituted alkyl, alkenyl alkinyl, aryl or aralkyl group comprising 1, 2, 3, 4, 5, 6, 7 or 8 carbon atoms. Preferably, if R is an alkenyl group, it comprises at most 3, 2 or 1 double bonds depending on the length of the alkenyl group. Preferably, if the hydrophobic moiety is an alkinyl group, it comprises at most 2 or 1 triple bonds, depending on the length of the alkinyl group. The alkyl, alkenyl or alkinyl group can be of a linear, a cyclic or a branched chain group. If it is of a branched chain alkyl, alkenyl or alkinyl group, preferably the one or more side chains of the branched chain is/are separately and independently from each other a methyl or and ethyl group. If it is a cyclic group, it may be a substituted or unsubstituted cycloalkyl or a cycloalkenyl group, e.g. a cyclobutyl, cyclopentyl, cyclohexyl, cycloheptyl or cyclooctyl group, or any unsaturated derivative thereof.

If R is a substituted alkyl, alkenyl alkinyl, aryl or aralkyl group the substituent can be any known substituent which does not disturb the formation of H bonds with the anionic surfactant in the micelles and which does not disturb solubility of the cationic complexing agent in the protic solvent, preferably water. Preferably, one or more of the substituents is/are a hydrogen donor moiety capable of forming a hydrogen bond. For example, the one or more substituent(s) is/are amino or imino group(s) or a group comprising one or more amino or imino groups.

In a preferred embodiment, if R is an alkyl, alkenyl alkinyl, aryl or aralkyl group comprising 2, 3, 4, 5, 6, 7 or 8 contiguous carbon atoms, then R′ or R″ or both is or are, independently from each other a substituted or unsubstituted alkyl, alkenyl alkinyl, aryl or aralkyl group comprising at most 6, 5, 4, 3, 2 or 1 contiguous carbon atoms, preferably at most 4, 3, 2 or 1, more preferably 2 or 1 carbon atoms or a —NH2, OH or H group. Highly preferably, R′ or R″ is in this case is a methyl, ethyl, propyl, isopropyl, —NH2, OH or H group. Highly preferably, R′ and R″ are different. Further preferred cationic complexing agents are, for example, compounds comprising the cation of general formula (II):
R—NH—C⁺(NH2)-NH—R′, (II)

wherein

R and R′ are H or substituted or unsubstituted alkyl, alkenyl, alkinyl or aryl group or, optionally, alkyl, alkenyl, alkinyl or aryl group substituted with one or more amino or imino groups or with a group comprising one or more amino or imino groups, or R and R′ are substituted or unsubstituted amino group; and R′ and R″ are identical with or different from each other.

In a preferred embodiment, one or two, preferably only one of R and R′ in formula (II) is or are a substituted or an unsubstituted alkyl, alkenyl alkinyl, aryl or aralkyl group comprising 1, 2, 3, 4, 5, 6, 7 or 8 carbon atoms, eg 2, 3, 4, 5, 6, 7 or 8 contiguous carbon atoms. Preferably, if R and/or R′ is an alkenyl group, it comprises at most 3, 2 or 1 double bonds depending on the length of the alkenyl group. Preferably, if the hydrophobic moiety is an alkinyl group, it comprises at most 2 or 1 triple bonds, depending on the length of the alkinyl group. The alkyl, alkenyl or alkinyl group can be of a linear, a cyclic or a branched chain group. If it is of a branched chain alkyl, alkenyl or alkinyl group, preferably the one or more side chains of the branched chain is/are separately and independently from each other a methyl or and ethyl group. If it is a cyclic group, it may be a substituted or unsubstituted cycloalkyl or a cycloalkenyl group, e.g. a cyclobutyl, cyclopentyl, cyclohexyl, cycloheptyl or cyclooctyl group, or any unsaturated derivative thereof.

If R is a substituted alkyl, alkenyl alkinyl, aryl or aralkyl group the substituent can be any known substituent which does not disturb the formation of H bonds with the anionic surfactant in the micelles and which does not disturb solubility of the cationic complexing agent in the protic solvent, preferably water. Preferably, one or more of the substituents is/are a hydrogen donor moiety capable of forming a hydrogen bond. For example, the one or more substituent(s) is/are amino or imino group(s) or a group comprising one or more amino or imino groups.

In a preferred embodiment, if R is an alkyl, alkenyl alkinyl, aryl or aralkyl group comprising 2, 3, 4, 5, 6, 7 or 8 contiguous carbon atoms, then R′ is a a substituted or unsubstituted alkyl, alkenyl alkinyl, aryl or aralkyl group comprising at most 6, 5, 4, 3, 2 or 1 contiguous carbon atoms, preferably at most 4, 3, 2 or 1, more preferably 2 or 1 carbon atoms. Highly preferably, R′ in this case is a methyl, ethyl, propyl, isopropyl, —NH2, OH or H group.

In a preferred embodiment in either formula (I) or formula (II) at least one of R′ and R″ is a H or both of R′ and R″ are H.

In a further preferred embodiment the cationic complexing agent comprises several, preferably at least three, four or five, complexing groups. Preferably, the complexing groups are separated by a spacer moiety comprising at most 10, preferably at most 8, 7, 6, 5, 4, 3, 2 or 1 contiguous carbon atoms. For example, the complexing agent is any of the compounds selected from the compounds of general formulae I or II, comprising multiple complexing groups. Preferably, in general formula I one or more, preferably one or two, or one of R, R′ and R″; or in general formula II one or two, or one of R, R′ is/are a group comprising one or more secunder amino groups and/or one or more guanidino groups. In this case any of the moieties or each of the moieties between N atom of formula (I) or formula (II) and the next secunder amino group or the next guanidino group, or between any or each of the secunder amino groups or the guanidino groups is/are a substituted or an unsubstituted alkyl, alkenyl alkinyl, aryl or aralkyl group comprising at most 1, 2, 3, 4, 5, 6, 7 or 8 contiguous carbon atoms or 1, 2, 3, 4, 5, 6, 7 or 8 carbon atoms. Preferably, if R and/or R′ is an alkenyl group, it comprises at most 3, 2 or 1 double bonds depending on the length of the alkenyl group. Preferably, if the hydrophobic moiety is an alkinyl group, it comprises at most 2 or 1 triple bonds, depending on the length of the alkinyl group. The alkyl, alkenyl or alkinyl group can be of a linear, a cyclic or a branched chain group. If it is of a branched chain alkyl, alkenyl or alkinyl group, preferably the one or more side chains of the branched chain is/are separately and independently from each other a methyl or and ethyl group. If it is a cyclic group, it may be a cycloalkyl or a cycloalkenyl group. In a preferred embodiment the cationic complexing agent is selected from tri-, tetra- or pentaamino alkanes, tris(2-aminoethyl)amin, tetraethylene-pentamine, spermine or spermidine.

Preferably, the meaning of one or more of R, R′ and R″ in the general formula I, or one or more, preferably one, of R and R′ in the general formula II is a group of general formula III as shown below:
—[(CH₂)ₓ—NH—]ₙH, (III)

wherein,

n is any integer from 1 to 10, preferably n is 1, 2, 3, 4, 5, or 6,

x is any integer from 1 to 8, preferably 1, 2, 3, 4, 5, or 6, and x can be identical or different in each of the repeating units.

Thus, the repeating units can also be identical or different in general formula III.

Preferably, n is 2, 3 or 4.

Preferably, x is 2, 3, 4 or 5, highly preferably x is 3 or 4.

According to a preferred embodiment of the invention, the meaning of R in any of general formulae I and II above is a group of general formula IV as shown below:
—(CH₂)ₓ₁—NH—(CH₂)ₓ₂—NH—(CH₂)ₓ₃—NH₂, (IV)

wherein, x1, x2 and x3 is 2, 3, 4 or 5, independently from each other.

According to a further preferred embodiment of the present invention, the meaning of R of any of general formulae I and II above is a group of general formula IV as shown below:
—(CH₂)ₓ₁—NH—(CH₂)ₓ₂—NH₂, (IV)

wherein, x1 and x2 is 2, 3, 4 or 5, independently from each other.

In further preferred embodiments in general formulae I or II one or more, preferably one or two, or one of R, R′ or, in case of formula (I), R″ is selected from the following groups: 2-benzimidazole, —O(O═)C—CH3, —(CH2)4-NH2, —(C6H5), —(CH2)4-NH—C(═NH)NH2, —(CH2)3-CH(NH2)-C(═O)OH, —(CH2)3-CH(NH2)-C(═O)O—CH2-CH3, —(CH2)3-CH3, —C6H10-NH2, —(CH2)6-NH2, —(CH2)2-N—(CH2-CH3)2, —CH2-CH2-OH, —CH2-CH3, —(CH2)2-NH2, —(CH2)2-O—(CH2)2-O—(CH2)2-NH2, —NH2, —OH, —(CH2)-CH(CH3)2, —C(═NH)—NH—(CH2)6-NH—C(═NH)—NH—C(═NH)—NH—(C6H4)-Cl, —(C6H5), —(CH2)3-NH—(CH2)4-NH—(CH2)3-NH2, —(CH2)3-NH—(CH2)4-NH2, —(CH2)2-NH—(CH2)2-NH—(CH2)2-NH—(CH2)2-NH2, —CH2-CH3, —C—(CH2-OH)3, —CH2-CH2-N(—(CH2)2-NH2)2.

In the method or use of the invention, the micelles can be conventional micelles, wherein the hydrophilic moiety of the surfactant molecules are located on the surface of the micelles, and preferably the colloid system is an oil-in-water system in which the polar, protic solvent is present in a continuous phase. However, the micelles of the invention can also be reverse micelles, in which the hydrophilic moiety of the surfactant molecules are located inside the micelles, and preferably the colloid system is of a water-in-oil type. Either of the two types of micelles can be used in various applications.

Below certain preferred methods and applications are disclosed.

According to an embodiment of the present invention, contaminants incorporated in or bound to the micelles that are present as a self assembling colloid system, e.g. a contaminants forming a complex with the micelles, are eliminated from an aqueous solution, wherein

micelles comprising the contaminants or micelles forming complex with the contaminants are produced in the aquous solution by the addition of the anionic surfactant

phase-separation is effected by using a cationic complexing agent, the micelle-rich phase is separated, and the procedure is repeated until a sample sufficiently free of contaminants is obtained.

The contaminant to be removed can be e.g. an apolar substance, or a polar substance capable of associating with micelles, such as an amide-comprising polymer, preferably a polypeptide or a protein.

Preferably, the polypeptide or the protein is separated from a solution containing nucleic acids, thereby the nucleic acid is produced in a purified form.

In a further preferred embodiment a nucleic acid is isolated from a sample comprising a mixture of nucleic acids, by adding an enzyme degrading the undesirable nucleic acid(s) to the sample, then, after the nucleic acid(s) have been degraded, removing the proteins from the sample via phase-separation, and optionally, the desired nucleic acid(s) is/are obtained from the equilibrium solution. Preferably, the nucleic acids are recovered by using the phase separation method of the invention (e.g., by polymolecular complex coacervation).

According to a preferred variation of an embodiment, the biological sample is lysed, the amount of anionic surfactant necessary for removing the protein content and—optionally—the amount of anionic surfactant necessary for removing the lipid content is determined, and the amount of anionic surfactant and cationic complexing agent, calculated on the basis of the above determination is added to the sample and, optionally, the sample is homogenated and/or phase-separation is facilitated based on density differences, e.g., the sample is centrifuged and/or the temperature and other conditions (e.g., pH, ionic strength, and dielectric constant) appropriate for phase-separation are adjusted, the phase-separation is allowed to proceed, then the micelle rich phase comprising the proteins and, optionally, the micelle rich phase comprising the lipids is separated from the equilibrium solution, and optionally, the nucleic acids are obtained from the equilibrium solution. The micelle rich phase is preferably a coacervate.

According to a further preferred embodiment of the invention, nucleic acids are recovered or separated by the use of the phase separation method of the invention, or optionally, are separated by a selective separation method.

Expediently, in aqueous solution, a coacervate comprising micelles that bind nucleic acids are formed by the addition of the anionic detergent and the cationic complexing agent to a nucleic acid containing system and, optionally, the formation of coacervate is initiated, then the coacervate enriched in micelles is separated.

Preferably, the nucleic acid containing coacervate is separated from the equilibrium phase by using a physical isolation method (such as sedimentation, centrifugation, filtration, spooling onto a glass rod, etc.).

The nucleic acids are recovered from the isolated coacervate.

According to a preferred embodiment, the coacervate is brought to solution by, for example, changing the ionic strength and/or dielectric constant of the solution by the addition of salt and/or an alcohol, then under suitable conditions phase separation, preferably, coacervation is initiated that allows the nucleic acids to remain in the equilibrium solution. Phase separation is preferably initiated by increasing the dielectric constant of the solution, e.g. by decreasing the concentration of the alcohol and increasing or essentially maintaining the ionic strength. Nucleic acids can be easily separated from the micelle-rich phase and, optionally, from other contaminants by using any suitable method.

According to a further embodiment, when the coacervate has been brought to solution, the nucleic acids are precipitated with alcohol. Preferably, after removing the micelle-rich phase free of nucleic acids, the nucleic acid is precipitated from the equilibrium phase with alcohol.

Preferably, the cationic complexing agent comprises multiple, preferably at least three, four or five complexing groups. Highly preferably, the complexing agent is an amine and the cationic complexing agent is an at least trivalent polyamine, preferably having at least three, four or five amino or imino groups, such as a group according to the general formulae I or II, in which R is the group of the general formula III above.

For example, the complexing agent can be an alkane comprising more than one, e.g. three, four or five, amino or imino groups, such as tris(2-amino-ethyl)amine, spermine, spermidine or tetraethylene pentamine.

Highly preferably, the isolation of the nucleic acids is monitored by the aid of a marker, such as stain, e.g. protein stain.

Highly preferably, the phase separation of nucleic acid(s) is performed subsequently to the removal of other compounds. For example, the separation of nucleic acid(s) is performed subsequently to the removal of the proteins and/or apolar substances, such as lipids.

In the method or use of the present invention the anionic surfactant is preferably a sulphate or sulphonate comprising alkyl and/or aryl group, preferably SDS or SDBS.

In this embodiment, the anionic surfactant and the cationic complexing agent is preferably selected so that the formation of emulsion may be avoided; preferably, the system may form coacervates at the temperature of the given application or use, preferably at room temperature, and the density may differ from that of the equilibrium solution. More preferably, the anionic complexing agent and the cationic surfactant are, respectively:

a) SDS and agmatin,

b) SDS or SDBS and butyl-amine,

c) SDS or SDBS and isobutyl-amine,

d) SDS and cyclohexyl-amine or

e) SDS and guanidine.

Preferably, in the purification method of the present invention a cationic complexing agent comprising multiple complexing groups, such as agmatin (having an amine and a guanidine group), or any of the compounds of general formula I or II comprising multiple complexing groups, preferably the variant having a group as to general formula III as substituent is used, for example any of the following is used in itself or in combination with one or more further complexing agent(s): diamino alkanes, tris(2-aminoethyl)amin, tetraethylene-pentamine, spermine or spermidine.

In a further preferred embodiment, a substance incorporated in micelles or bound to micelles forming a self-assembling colloid system—e.g. a substance forming a complex with a micelle—is isolated from an aqueous solution as follows:

micelles comprising the target substance or bound to the target substance are produced in the aqueous solution by adding anionic surfactant,

phase-separation is effected by adding the cationic complexing agent, and

the micelle rich phase is separated.

According to this embodiment, preferably
the target substance incorporated in or bound to the micelles can be recovered from the micelles,
micelles comprising the target substance can be isolated from the micelle rich phase by their size, etc.

According to a preferable embodiment the substance to be isolated is a protein isolated from the coacervate comprising a mixture of proteins, by the differences in micelle sizes.

According to this embodiment preferably the cationic complexing agent and/or anionic surfactant comprises sulphate, preferably the cationic complexing agent is SDS.

According to a further preferred embodiment, the compound to be isolated is a nucleic acid, such as DNA or RNA. The nucleic acid might be single stranded or double stranded, circular or linear.

According to this preferred embodiment, the cationic complexing agent comprises several, preferably at least three, four or five, complexing groups. Highly preferably, the complexing group is an amino or imino group, and the cationic complexing agent comprises at least three, four or five amino or imino groups. For example, the complexing agent is any of the compounds selected from the compounds of general formulae I or II, comprising multiple complexing groups, preferably a variant comprising a group of general formula III as a substituent, such as any of the following: tri-, tetra- or pentamino alkanes, tris(2-aminoethyl)amin, tetraethylene-pentamine, spermine or spermidine.

According to a further preferred embodiment, at least two proteins, incorporated in the micelles or bound to the micelles, e.g. substances forming a complex with micelles composing a self-assembling colloid system, are isolated from an aqueous solution as follows:

anionic surfactant is added to the aqueous solution comprising the proteins, in an amount less than necessary to reach saturation of the proteins,

the cationic complexing agent is added to the solution in excess amount relative to the amount of anionic surfactant, thereby coacervation is effected,

a partition of the proteins, based on differences of their isoelectric points, is effected by adjusting the pH of the solution,

the desired protein is isolated by phase-separation, on the basis of phase-partition.

Separation can be performed by, e.g., gel-filtration, hydrophobic chromatography, or ion-exchange chromatography. In this case, the anionic surfactant preferably comprises sulphate ion and the surfactant may be e.g. SDS.

In the methods of the invention, attention should be paid to the adjustment of the temperature, pH, ionic strength, dielectric constant and/or other parameters of the system, if necessary. Optionally, precipitation, coacervation or solution, or the binding or non binding of the associate to the coacervate or to the precipitate can be provided by changing these parameters. Based on the teaching provided herein a person skilled in art can adjust these parameters in consideration of the given use.

According to a further preferred embodiment, following the phase-separation, the micelle rich phase comprising the target substance or micelles bound to the target substance is converted into micro- or nanodispersion or into micro- or nanocapsules.

The phase-separation can be, e.g., coacervation and coacervate obtained by coacervation is emulgeated in a suitable solvent, then microcapsules or nanocapsules are formed from the emulsion.

In a further preferred embodiment, micelles in microcapsules form micelle crystals.

It is advisable to select a system that coacervates above the temperature of the desired or proposed application, thereby producing coacervate. Thereafter, microcrystalline dispersion or capsules are obtained by cooling the coacervate.

The micro- or nanocapsules may contain, for example, stains or dyes or biologically active substances, such as test compounds, active agents, medicaments or cosmetics, or, for example, proteins, peptides, nucleic acids, etc.

The active agent containing micro- or nanocapsules might be used, for example, to pack, administer or target active agents or test compounds.

If the micro or nanocapsules contain nucleic acids, according to certain embodiments they can be used for introducing the nucleic acids into living organisms, e.g. to cells, or for transforming the organisms, or they can be applied as vectors.

According to a highly preferable embodiment, the micelle-rich phase of the invention can also be used for storing further substances enclosed in the micelles or bound to the micelles; such further substance might be, for example, any of the following: proteins, nucleic acids or apolar substances, such as lipids, stains or dyes, medicaments, cosmetics, synthetic polymers, etc.

Provided that the mixture of substances, such as a coacervate or a precipitate, is also stable together with the equilibrium solution, it can be packed, stored or transferred in a container. The removal of the equilibrium solution may lead to the dessication of the phase enriched in micelles, however, in certain embodiments this does not present a problem; in these cases the storing is possible even after removing the equilibrium solution.

In a further variation, subsequently to the completion of the methods of the invention, the mixture of the invention which is suitable for maintaining the micelle structure is covered or coated with a medium of constant composition. In the mixture of the invention the micelles may contain further substance enclosed in micelles or bound to the micelles, such as a substance which forms complex with the micelles. Highly preferably, following the phase-separation, the phase enriched in micelles (preferably coacervate) is separated from the equilibrium solution, and coated with a cover medium (layer) having an ionic strength not higher than the ionic strength of the equilibrium solution and/or having a dielectric constant not lower than the dielectric constant of the equilibrium solution.

Preferably, the covering medium is a gel or the covering medium is a solution covered with solid material.

Preferably the anionic complexing agent and the cationic surfactant is, respectively:

a) SDS or SDBS and guanidin,

b) SDS or SDBS and anilin, and preferably the encapsulated substance is a protein,

c) SDS and ethylene-diamine,

d) SDBS and cyclohexyl-amine or arginine.

e) SBDS or SDS and multivalent amine, preferably an amine comprising at least three amino group, such as spermine, spermidine, tetraethylene pentamine; the encapsulated substance preferably is a nucleic acid.

In this embodiment, the complexing agent preferably carries functional groups capable of forming crosslinks, and after phase-separation the micelles are reacted with the crosslinker so that the wall of the micelles is stabilized.

In a further preferred embodiment of the invention the phase separation is coacervation and the micelle rich phase is emulsified in an appropriate solvent so that micro- or nanoemulsions are formed.

According to a further aspect the invention relates to kits for performing the methods or uses of the invention. The kit of the invention comprises at least

an anionic surfactant, and

a small molecular cationic complexing agent which, along with ionic interaction, are capable of forming complexes via a further secondary interaction. Additionally, the kit comprises any defined component required to carry out any of the methods or uses of the invention.

### DEFINITIONS

By “structural component” of a micelle is meant herein a certain molecular material that plays an essential role in constructing micelles, determines the features of the micelle and without it the structural characteristics of a particular type of micelle cannot be formed or the structure formed would be basically different. The entirety of the structural units of a micelle can form the given type of micelle in an appropriate solvent and it can do so even without the involvement of other components.

By “anionic surfactant” is meant herein a characteristically amphoteric molecular material that can form micelles in themselves in an appropriate solvent. The molecular material has an apolar and a polar portion, and in a solution of protic solvent, preferably in aqueous solution, it carries negative charge on its polar part in a given pH-range. The anionic surfactant can be present in salt- or ionic form.

By “cationic complexing agent” is meant herein a small molecular substance that carries positive charge when it is in a solution of protic solvent, preferably in an aqueous solution, and in a given pH-range, and it can form ionic interactions as well as other secondary bond(s) with an appropriate anionic surfactant. Besides ionic bonds, there are secondary bonds, such as hydrogen bonds, ionic-dipol interactions, dipol-dipol interactions, induced ionic dipol interactions, where the binding effect roots in the resultant of the binding forces. Preferably, the cationic complexing agent can form at least one hydrogen bridge bond with the anionic surfactant. Preferably, the cationic complexing agent contains a hydrogen donor group, most preferably, an ammonium group or a guanidinium group in protonated form. The term “cationic complexing agent” includes both the deprotonated and the protonated forms of these substances, such as dissolved protonated form or their salts. Preferably, the cationic complexing agent is not a surfactant.

By “complexing group” is meant herein the complexing group of the cationic complexing agent or the anionic surfactant.

A molecule “smaller than colloidal size” is a molecule which does not reveal colloidal characteristics. Alternatively, a molecule “smaller than colloidal size” is a molecule any length of which is not larger than 1 nm.

A “non-colloidal size complexing agent” is a complexing agent the molecules of which can not form micelles or micellar colloids in a protic solvent e.g. an aqueous solvent or, alternatively, the molecules of which are not larger than 1 nm in size.

By a substance of “small size molecule” is meant herein a substance composed of molecules smaller than those of colloidal size or the molecular mass of substances of small size molecules is up to 1000, more preferably up to 500, 400, most preferably up to 300 or 200.

By “capsule” is meant herein a cluster of molecules with complex structure that contains micelle(s) and other substances enclosed in the micelles, and it has a “shell” isolating the internal part of the capsule from the external environment, the shell is sufficiently stable to block the release of the entrapped material to the environment for the time required by the given application. Provided the capsule is composed of a single micelle containing anionic surfactant and a cationic complexing agent as structural components, it is possible that the capsule shell is substantially identical with the network of anionic, polar groups and cationic complexing agents flanking the micelle, or at least this network plays an important role in the formation of the shell. Optionally, crosslinkers, polymers, detectable molecules, epitopes, receptors, groups for binding conjugates can also be involved in forming the capsule shell.

By “microcapsule” is meant herein a capsule with the diameter ranging between 1 μm and 1000 μm. The “nanocapsule” is a capsule with a diameter ranging from 1 nm to 1000 nm. By “phase separation” of a colloid system containing micelles is meant herein a process where, due to the change in the thermodynamic state, the originally homogenous solution separates into two phases, one rich in micelles and the other one called equilibrium solution. The phase rich in micelles can be solid or liquid. If the phase rich in micelles is a solid phase, it differs from the precipitate in that, in this case, the phase contains micelles and its structure reveals ordered parts, preferably it forms micelle-crystals. When the phase rich in micelles is a liquid, the process is called “coacervation”.

By a “self-assembling” colloidal system is meant herein a system that—under given circumstances—reaches thermodynamically stable or metastable state without any external influence.

### SHORT DESCRIPTION OF THE FIGURES

FIGS. 1.a. and 1.b. show the microscopic picture of the product micelle-crystallized by freezing with liquid nitrogen. The hair showed in FIG. 1.a. has a diameter of approximately 80 μm, thus the diameter of the micelles ranges 5-10 μm. Since, in order of magnitude this size is several times higher than the original one, the micelles are either of vesical type that contain solvent, or they form aggregates of smaller micelles.

FIG. 2 shows regular guanidinium-dodecyl-sulphate crystals crystallized in the excess of SDS. Compared to the micelle crystalline structure (FIG. 1) the difference is evident.

FIG. 3 shows a mixture of 100 mg/ml BSA and 0.5 M SDS (1:2, v/v) (200 μl); 1% Brillant Blue R-250 stain (CBB, 20 μl); and 1M Guanidinium-chloride (400 μl, no salt, pH 7.0). The coacervation takes places at 60° C. Practically all of the CBB-stained BSA can be seen in the coacervate.

FIG. 4 shows a dispersion prepared by liquid nitrogen freezing and vortexing the coacervate of FIG. 3. The dispersion contains micelle crystalline structures stained with CBB (“protein capsules”), the microscopical picture of which can be seen in FIG. 4.b.

FIG. 5 shows an electromicroscopical picture of associates. They were prepared by the careful centrifugation of the dispersion showed in FIG. 4, then the smaller particles including the associates, which remained in the supernatant, were transferred to microscopy slides. After being dried, the slides were processed as usual. It can be seen from the size that the sizes of structures are in the nanometric range. The rounded structures clearly indicate that the units are not regular crystals but micelles.

FIG. 6 shows a diagram explaining simple coacervation.

FIG. 7 shows a diagram explaining complex coacervation.

FIGS. 8.a. and 8.b. show the process of the polymolecular complex coacervation according to the invention.

FIG. 9 shows the possible interactions of the dodecyl sulfate, as anionic surfactant, and two types of cationic complexing agents: guanidinium ion and butyl ammonium ion (hydrogen bridge and hydrophobic interactions).

FIG. 10 shows a DNA-containing clew-like coacervate stained with CBB.

FIG. 11 shows a DNA-containing coacervate stained with CBB after centrifugation; the coacervate forms a viscous block adhered to the bottom of the container. The coacervate is covered with agarose gel.

FIG. 12.A shows the agarose gel electrophoresis image of plasmid DNA obtained by the nucleic acid isolation method based on complex coacervation. The control was a lambda DNA marker (M) digested with Hind III restriction enzyme

FIG. 12.B shows the checking of isolated DNA that was precipitated with alcohol. DNA was dissolved in 5 μl TE (pH 7.5) buffer and denoted by (A), or (B1). Samples (A), (B1) and (B2) are checked by agarose gelelectrophoresis. Control: 1 μl of the original solution, i.e. the DNA content of sample (A), (B1) and (B2) theoretically corresponds to 1.5-times, 15 times or 1.33 times of the DNA content of the control.

### A DETAILED DESCRIPTION OF THE INVENTION

It is generally accepted that the phenomenon of coacervation is closely associated with the ionic bond and other intermolecular interactions as well as with the characteristics of dissolution, hydration and features of water, as solvent. However, other polar, protic solvents—such as methyl- or ethyl alcohol, glycol or acetic acid—theoretically can also be used under certain circumstances. Since our experiments were performed in aqueous medium, usually water is mentioned herein. However, we should bear in mind that by creating suitable conditions (pH, ionic strength), water can be replaced by other solvents, too.

In solid materials, the ionic bond represents a fairly strong interaction between the oppositely charged ions. As to its order of magnitude, its strength is equal to the covalent bond. However, in aqueous solutions the degree of the interaction hardly exceeds the energy of the thermal motion. This is due to the fact that every ion dissolved in water is surrounded by several water molecules, and due to the ionic-dipole interaction between the water molecules and the ions, the strength of the ion-ion interaction, which changes with a power of 2 of the distance, decreases.

Ions and poly-ions dissolved in water are always surrounded by a dynamically changing, still adherent water shell. This water shell, i.e. the hydration of molecules, inhibits the formation of other interactions between molecules. The decrease or elimination of this adherent water shell forms the basis of coacervation. In the case of simple coacervation, the water shell surrounding the charged colloid is eliminated or decreased by the addition of hydrophilic substances (such as salt, alcohol). As the colloid particles get closer to each other, interaction can occur, which results in the formation of a separate phase. The complex coacervation differs from this process. In this case the ionic interactions of a polycation and an equally charged polyanion are added up and the resultant binding force overcomes the interaction between the individual ions and the dipoles of the water, which results in the diminishing of the amount of ion-bound water; the water shell disappears and the colloid particles can get closer to each other which enables the formation of associative interaction among the colloid complexes. Finally, phase separation occurs. The precondition of two opposite charges being quenched is that the ions should get sufficiently near each other, i.e. the flexibility of the molecules is essentially important in the occurrence of complex coacervation. As for the state of the art, only linear proteins can induce complex coacervation, whereas the same cannot be done by globular ones constituting the major part of proteins.

In the phenomenon found by the present inventor, one of the components is of a colloidal-sized associative poly-anion, while the other is a small molecular weight complexing agent applied in a number corresponding to the charge of the poly anion. Although a small molecular weight component is used in the method, it is far from being a simple coacervation process, as it is not based on dehydration. As a contrast to simple coacervation, it is not necessary to apply small molecules in great excess. The phenomenon also differs from the complex coacervation, where the process is based on the interaction of two macromolecules. In the method of the invention, not only the size of the molecules differs, but also the type of the interaction. In the conventional complex coacervation, the stabilization of each of the ion pairs—i.e. the diminishing of the interaction between the water dipoles and the ion—is primarily driven by the fact that the given ion pair is covalently bound to adjacent ion pairs. Thus, the electrostatic interactions of the macromolecule are added up. Although in the phenomenon found by the present inventor charge neutralization also plays a significant role, the stabilization of ion pairs, i.e. surmounting the interaction between water dipole and the ions, is provided by the complexing effect of the small molecules bound to the colloid particle, i.e. the individual ionic bonds, together with other intermolecular interactions. To distinguish the conventional complex coacervation from the phenomenon presently discovered, it was decided to call it polymolecular complex phase separation or—when two liquid phases are formed—polymolecular complex coacervation. The inventors found, nevertheless, that not only the so-called coacervation, i.e. the formation of two visible liquid phases, can be applied industrially, but on the basis of the same principle, a solid phase concentrated in micelles and an equilibrium solution can be formed, and the two phases (the one concentrated in micelles and the equilibrium solution) can constitute disperse systems, i.e. suspensions. Similarly, emulsions can be prepared.

As a rule, the system according to the invention has self-assembling ability. The whole polymolecular system is held together by a network of ionic and secondary bonds having the energy level that hardly exceeds that of the thermal motion. In this system, due to its complexity, —with the exception of covalent and metallic bonds—all of the known types of chemical bonds may play a role. The most important interactions are as follows: ionic, hydrogen, van der Waals, hydrophobic, ion-dipole, dipole-dipole, etc. Since the system seeks an energy minimum, the identical components are stabilized by a network of secondary, reversible interactions between each other and also with the different components, i.e. are arranged by self-assembling. The whole system is driven by the formation of complex micelles which determine the micro-environment to which the associates also adapt themselves.

The hydrophobic (apolar) tail and the hydrophilic (polar) head are common characteristics of surfactants. The group of anionic surfactants (the hydrophilic head of the surfactants carries anionic charge) is mainly composed of sulfonic acid derivatives and sulfate esters. The two most prominent representatives of this group are the sodium dodecyl sulphate (SDS) and the sodium dodecyl benzenesulphonate (SBDS). The first has mainly scientific significance while the other is primarily used in industrial applications. It is well known that these compounds can interact with several substances (such as fats, oils, proteins, contaminants, etc.); this is one reason why we use them as detergents or cleaning agents. The emulsifying nature of the surfactants is based on the fact that they surround the fat or oil drops in such a way that while their hydrophobic tail interacts with lipids, the hydrophilic head turns to the water phase. Thus the drops remain in solution.

One of the important features of the surfactants is that the number of their individual molecules in aqueous solution cannot exceed a certain limit. By increasing the amount of the surfactants, the molecules start to form typical aggregates. The aggregation number is the number of aggregated monomers. In the case of SDS, for example, this number is 62 (molecular weight ˜18.000). These aggregates are called micelles, the concentration limit is the critical micellar concentration (CMC). The CMC is characteristic of each type of surfactants. The temperature at which all the three surfactant phases (crystals, micelles, and monomers) are at equilibrium is called the Kraft point.

In the micelles the surfactant molecules form a characteristic spherical or elliptical shape where their hydrophilic part faces to the water. Their typical size is 3-6 nm. Depending on the concentration of the surfactants, temperature, counterion, etc., micelles can form rod, double layer, etc. shapes. The micelles are not static formations, their components are continuously changing and transforming.

In apolar organic solvents reverse micelles can also be formed, and the hydrophobic part of these micelles faces outward and the polar, protic solvent, preferably aqueous solution, is entrapped inside. Polar molecules that do not form complex with the structural components of the micelle can also be entrapped in the micelles.

In the field of research SDS is the most popular anionic surfactant. The interaction between SDS micelles and proteins has been discussed in numerous publications. According to the most widely accepted “necklace” model, the SDS micelles are seated on the protein chain as beads of a necklace. In practice, this interaction is most widely utilized in the denaturing electrophoresis of proteins. In saturated SDS solution (to achieve this, the necessary amount of SDS is 2.2-2.5-fold higher than the weight of the protein), proteins bind the SDS in an amount proportional to their molecular weight. The highly stable protein-SDS complex is maintained during the electrophoresis. Since the electrophoretic motility depends on the total number of negative charges of the protein-SDS complex, the molecular weight of the proteins can be determined on the basis of the migration length.

The SO₃⁻ group of both the sulfonic acids (R—SO₃⁻) and the sulphate esters (R—O—SO₃⁻—) carries a negative charge and has a total of six non-binding electron pairs distributed on the three oxygen atoms, respectively. As a H acceptor, either of the two non-binding electron pairs of the oxygen atom can form hydrogen bonds. This atomic group can interact with several water molecules, i.e. the molecules carrying such a group are well hydratated. This is why this type of molecules is considered to be the best surfactants. The characteristics of the micelles, thus of the surfactants, are determined not only by their hydrophilic part but also by the hydrophobic part, additionally, the property of the two parts together, i.e. the hydrophilic-hydrophobic balance should be taken into account. Since small differences can cause significant changes, a great number of different characteristics can be formed and these substances have a wide range of application.

In the micelles, the adjustment of the surfactant molecules is determined partly by the structure of the hydrophobic portion (branched aliphatic, aromatic, etc.) and partly by the repulsive force between the anions. As a matter of fact, on the surface of the micelle the area taken by the individual surfactant molecules is rather small and this topological fact determines the size of molecules which are capable of neutralizing the negative charges of the micelle. Only uncharged molecules can coacervate. However, the neutralization of the charge is only a necessary but not sufficient condition of coacervation. To achieve coacervation, it is also necessary to exclude the hydrating water molecules. The hydrogen bond is the strongest among the secondary or intermolecular interactions. As we have already seen above, the SO₃⁻ group can form six hydrogen bonds, thus it is reasonable to select a cationic molecule suitable for neutralizing the negative charges from among substances having hydrogen donor property. It is evident that the water molecules individually form hydrogen bridges with the oxygen of the SO₃⁻ group. If appropriate complexing agent is selected, a single molecule can contain several hydrogen donor atoms, so the bonds of covalently bound hydrogen donor atoms can be summarized at molecular level. The ionic bond supplemented with hydrogen bonds, and optionally further strengthened by free energy changes originating from other interactions, can ensure both the exclusion of the hydrate shell surrounding the hydrophilic portion of the surfactant molecules and the immobilization of the complexing agent on the surface of the micelle. A further interaction strengthening the cationic bond of the cationic complexing agent can be also hydrophobic interaction. The effect that orders the surfactant molecules into micelles demonstrates the strength of this type of interaction. The internal lypophilic environment of the micelles is suitable for including molecules or molecular parts of similar type. Provided the cationic complexing agent together with one or more such groups can dissolve inside the micelle, the micelle anchors the complexing agent on its surface.

As to the complex formation according to the invention it is essential that water molecules solvating the micelle formed by anionic surfactants are to be excluded as far as possible from the solvate shell of the micelle. The cationic complexing agent, besides neutralizing the charge of the micelle, should occupy the hydrogen acceptor groups as these otherwise can bind a large amount of water molecules. The cationic complexing agent can not be a typical surfactant or it can not have a pronounced surfactant character, as otherwise a strong hydrophobic interaction would cause this cationic surfactant incorporate into the micelle with its apolar tail forming a mixed micelle. In this case the cationic molecule, even if it has a hydrogen donor group, can not form a hydrogen bond with the anionic surfactant as the hydrogen donor and the hydrogen acceptor groups are not positioned in an appropriate angle.

The cationic complexing agent may have a certain amphipatic character. A weak hydrophobic interaction to an extent which does not prevent the formation of a hydrogen bond is not disadvantageous. Such a limited hydrophobic interaction may even advance or fortify coacervation by hydrophobizing the complexed micelle (excluding hydrating water molecules).

Based on the above considerations, preferred complexing agents suitable for coacervation of micelles formed by anionic surfactants (or colloid systems consisting of such micelles which may materials being in close contact with the micelles, forming association complexes therewith), can be found among compounds containing ammonium or guanidinium groups, or their derivatives.

The formation of complexes is a very complicated process and the different theories can only explain the formation of the already existing complexes, however these theories cannot precisely predict which compound will work as a complexing agent in a given system. The theories of the invention are aimed at making the processes more comprehensible, however, any deviation from reality will not limit the practice of the invention. On the basis of complexing micelles formed by SDS or SDBS, several conclusions can be drawn. By studying these compounds, certain similarities and differences can also be observed. In the case of SBDS micelles, the area occupied by each (the individual complexing agents) of the complexing agents is significantly bigger than that of the SDS. The sulfonic acid group can form significantly stronger bonds than those of the SDS. The hydrophobic interaction holding together the SBDS micelles is stronger than that of the SDS. The interaction between SBDS micelles and the anionic complexing agents is stronger than in the case of the SDS. Thus, the interaction of SDS micelles are “softer” while the interactions of SBDS micelles are “harder”. The fact, that in the polymolecular complex coacervation the complexing of the anionic, hydrophilic group is the determinant factor, is well demonstrated by the formation of polymolecular complex coacervation by using the Brillant Blue R-250 protein stain (a stain which although has two sulfonic acid groups and a big apolar portion, but not classified as a surfactant) and isobutyl-amine hydrochloride in our laboratory. On the basis of this result, we have concluded that the stain forms micelles and has an ampholytic character.

The differences between the complexing agents are extensively significant. Generally, compared with the guanidinium group, the members of the ammonium group can form weaker interactions. Regarding their structure, this phenomenon is quite understandable: while the members of the ammonium group (R—NH₃⁺) could have a maximum of 3 hydrogen donors, in the guanidinium group (R—NH—C⁺(NH₂)₂) the number of donors is five, or it reaches even six in the basic compound (C⁺(NH₂)₃).

The interaction between guanidinium chloride (or guanidine hydrochloride) molecules and the SDS or SBDS is very important from both theoretical and practical points of view. Considering the two groups [SO₃⁻ and C⁺(NH₂)₃] participating in the interaction, it is obvious that there is the theoretical possibility of ionic bonds and hydrogen bonds (in which all of the oxygens are engaged) to be formed between the guanidinium chloride and SDS or SBDS micelles (the fourth oxygen atom of the SDS is situated probably under the surface level of the micelle). It is well known that the formation of the hydrogen bond and its strength particularly depends on the angle between the donor, hydrogen and acceptor. Based on the observed phenomena and without performing instrumental measurements, the inventor presumes that this complete or near-complete interaction will be formed between the molecules.

Interesting phenomena can be observed when aqueous SDS solution and aqueous guanidinium chloride solution are combined. Right after the combination of the two solutions generally a precipitate is formed, however, sometimes only the viscosity of the solution increases significantly and the precipitation occurs only after minutes, particularly when the two compounds are combined in equimolar or near-equimolar amount. In the excess of SDS, the white precipitate is composed of long, transparent, regularly shaped, needle crystals, which are terminally closed in 45°. The melting point of the crystals is 146° C. In the excess of guanidine, besides the crystals, bunches of sphere-like particles and irregular, non-transparent agglomerates appear.

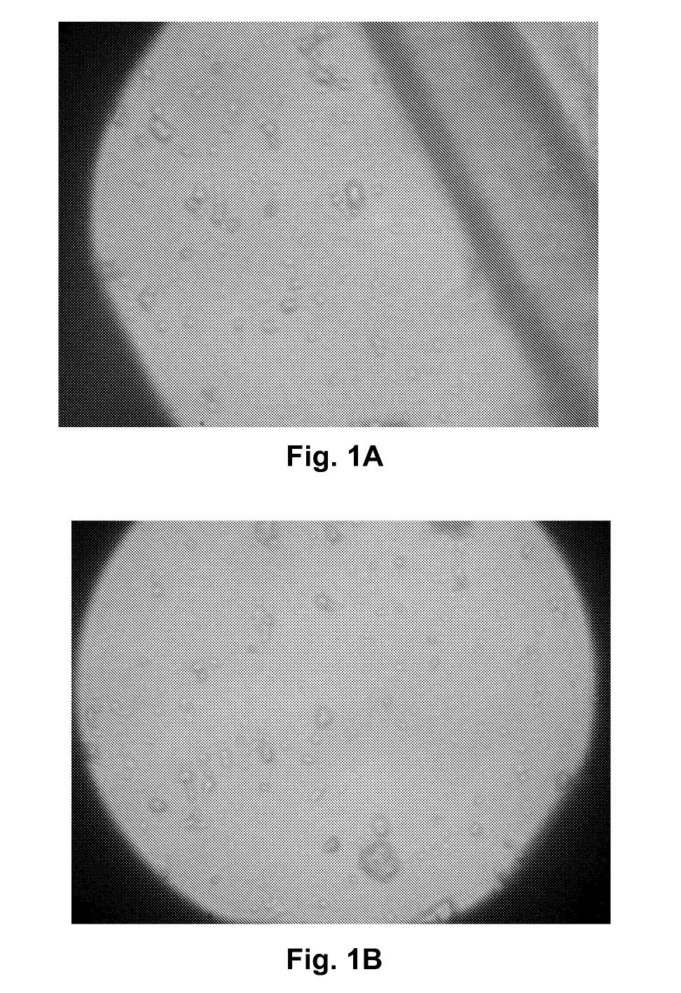

When the solution containing preformed precipitation was warmed in the presence of excess guanidine, the precipitate “melted” at about 55° C. and the solution separated into two liquid phases, i.e. coacervation occurred. (By being further warmed, the solution became one-phased again). When the solution separated into two phases was cooled quickly, the coacervate transformed into a white, amorphous, powder-like substance. By observing under microscope, the inventors found that the material was composed of adhering, smaller, globular particles with a size of a few micrometers (FIG. 1). The process is reproducible, i.e. the precipitate “melts” by being warmed to about ˜55° C., and the liquid separates into two phases.

The phenomenon described above is interpreted as follows: when the SDS and guanidinium chloride molecules are combined at ambient temperature, the guanidinium molecules interact with the SDS molecules. The micelles containing partly sodium ions and partly guanidinium counterions are instable. The guanidinium dodecyl sulphate molecules leave the micelles and form regular crystal lattice, while the sodium chloride remains in solution.

When warming the micelles in excess guanidinium, the bonds providing the solid or crystalline structure in the guanidinium dodecyl sulphate solution weaken and due to the hydrophobic interaction, the molecules form micelles. The guanidinium excess ensures that the guanidinium group neutralizes all the negative charges of the micelles. Due to the complexing of the guanidinium, the hydrate shell around the micelles reduces and interactions can occur between micelles (which process was previously inhibited by the hydrate shell). The micelles adhere or may even fusion, and due to the significant change in the hydrophilic/hydrophobic balance of the surfactant molecules caused by the process of complexing, the micelles can take different geometrical shapes. (The accurate shape and structure of the associate formed by complexed surfactant molecules can be determined only by instrumental measuring, however, the lack of this information does not hamper the utilization of the phenomenon). Finally, the adhered, badly solvated particles form a separate phase. The quick temperature-decrease “freezes” the instantaneous status of the micelle agglomerate. The components of the complex become immobilized and solid. The different interactions of the micelle and the complexing agent become delocalized, chemical bonds will be formed all over the surface and in the inner part of the micelle, and the metastable state of the micelle becomes stabilized. Quick cooling blocks the initiation of regular crystallization (which represents a lower energy level than that of the metastable state) that would cause the demolition of the micelle structure. The microscopical picture of the solid micelles or micelle agglomerates shows similar size, globular structures. The structures completely differ from the regular crystals, however, they are not shapeless, thus we cannot classify them as amorphous. Since in an ideal case, the SDS micelles are globular, to distinguish these structures from regular crystals, we can call them globular or spherical crystals. However, since their shape in effect is usually differs from a sphere—especially when they form association complex with a macromolecule, such as a protein, —generally they are called herein micelle-crystals.

The following finding supports our theory. Regularly crystallizing guanidinium sulphate and its micelle-crystalline (i.e. globularly crystallized) form was prepared. Under microscope, water drop was added to each type of crystals and the samples were allowed to dry. The regularly crystallized sample was recrystallized, i.e. after a preliminary reduction, regularly-shaped crystals with a size of larger than the original developed on the slide. The globularly crystallized material did not change during drying, the material retained its original form: macroscopically powder-like, microscopically small globular structures which adhered to each other more or less, and which constituted chains or bundles. The inventors think that while regular crystals form open-ended agglomerates susceptible to dissolution or crystallization, the micelle-crystals form close-ended agglomerates that are not susceptible to dissolution or crystallization.

Similar micelle-crystallizing phenomenon has been observed in the case of SDS and aniline hydrochloride system. The difference between the two crystals is even more apparent. While the SDS-anilide forms macroscopically large, needle-like crystals with a melting point of 121° C. and which, in polarized light beam, shows all the colours of the rainbow, the micelle-crystals obtained from the coacervate produce a non-transparent, amorphous mass. When micelle-crystals of coacervate origin are warmed up, above a temperature of about 90° C. more and more structures, which turn the plane-polarized light, appear in the amorphous agglomerate. When the melting starts at around 121° C. only regular crystals showing the colours of the rainbow in the polarized light can be found in the melt. We think, without limiting the invention, that the molecules forming the micelle-crystals rearranged from their metastable state to a stable state of regular crystalline structure.

The inventors presume, that a surfactant molecule can interact with several complexing agents and vice versa a complexing agent can interact with several surfactant molecules. In the case of micelle-crystallization it is possible for a condition like the above to develop, i.e. the molecules are arranged in a curved surface crystalline lattice. This is considered to be more probable than a one-to-one interaction, but its speculative nature should be emphasized. It is likely that the inclination for overcooling and the simultaneous solidifying are caused by the phenomenon that, one of the complexing agent molecules, being opposite to the detergent molecules in the liquid phase coacervate, “jumps in” a position wherein it fits in the detergents molecules; this site serves as a first “crystallization centre”, followed by further ones. It should be kept in mind that this is mere theory which by no means may restrict the scope of the invention.

The different compositions show a diverse picture. There is such a system which coacervates at 30° C., but forms micelle-crystals at ambient temperature. Other compositions coacervate at ambient temperature and form micelle-crystals by cooling. As a whole, complexing agents can be used for different purposes.

We were able to obtain polymolecular complex coacervation of micelles by using several complexing agents (Table 1). Different complexing agents can form interactions of different type and strength with each detergent molecule. Additionally, the complexing agents carry chemical groups with different characteristics and these groups significantly determine the surface characteristics of the complexed micelle (e.g. hydration with different level, chemical characteristic, interaction with other micelles, etc.). While traditional complex coacervation (where particles lacking an overall charge and consisting of weakly solvated associated macromolecules in pairs) a relatively minor diversity, depending on the quality of components, can be observed, polymolecular complex coacervation shows a highly varied picture. From systems separating into two clear phases within minutes to stable nanocolloidal systems several variations could be observed. A complexing agent that had already coacervated with a surfactant did not interact with any other surfactant, however, the viscosity of the solution may have increased incidentally, or contrarily, insoluble precipitate was formed. Sulphate or sulphonate groups provide sufficient water solubility necessary for the size of micelles that are composed of surfactant molecules. When complexes are formed through these groups the complexing agents containing apolar groups or even polar groups cannot provide hydrate shell with a size suitable for the dissolution of the colloid particles. However, surprisingly, we found that the micelles complexed with hydroxylamine, hydrazine or arginine hydrochloride show coacervation. It is possible, that the hydroxylamine or carboxyl groups of these compounds take part in the process of complexing.

Coacervation, i.e. when a solution separates into two phases is a macroscopical phenomenon. The phase separation is caused by the decrease of the solvatation of the colloid particles. In the polymolecular complex coacervation the surface characteristics of the colloid particles are determined by the complexing agents. Due to the diversity of the complexing agents, the complexed micelles can be of many different types, while in the case of the conventional complex coacervation, the chemical characteristics of the components are very similar. During coacervation the hydrate shell does not completely disappear, the complexed anionic portion will not become (it cannot become) as much apolar as the carbohydrogen chain of the surfactant molecule. Depending on the characteristics of the complex and the rate of complexation, the hydration rate of the complexed micelles may vary within a wide range. This phenomenon contributes to the diversity of the embodiment of the invention. When the hydrate shell is relatively fat, the interaction between the particles will be weaker, therefore, the phase separation, which is characteristic of the coacervation, will need a longer period of time to be completed, although charge neutralization will take part just as it happens in quickly separating systems. In this case the particles form stable emulsion.

The polymolecular complex coacervation of micelles can be carried out by using mixed systems, i.e. the mixture of complexing agents and/or the mixture of anionic surfactants. Additionally, the so-called heterogeneous micelles prepared by the addition of a little amount of non-ionic surfactants provide further possibilities of application. Compounds that have more, sometimes already two, complexing groups, which generally form insoluble precipitate cannot be used in themselves, but they can be preferably useful when mixed, in a little amount, with well coacervating complexing agents. The same is true of the complexing agents whose product is water-soluble. The complex coacervation of the invention is a polymolecular process, i.e. the micelles and complexing agents forming the colloid particles are small molecules and any of them can be part of a mixture. Considering merely the great number of anionic surfactants and the numerous complexing agents that initiate coacervation, the procedure provides practically endless variations. The characteristics of coacervated colloid particles can vary in a wide range and in a predetermined manner by changing the composition of the starting materials.

Besides the ratio and the concentration of the detergent and the complexing agent—as usual in the colloid systems—the most important parameters that affect the coacervation are the pH of the solution, the ionic strength and the salt composition of the solution and the temperature. The theoretical difference between conventional and polymolecular coacervation results in a different parameter-dependence of the two systems. Conventional coacervation takes place in a narrow pH range, where the charge of the macromolecule provides the charge compensation, the system is not sensitive to temperature changes, the concentration of the components can be changed in a rather narrow range and their ratio is fixed.

Quite to the contrary, polymolecular complex coacervation can be performed in a wide pH range. A condition of fundamental importance is that both the anionic and the cationic group should be in an ionized state. Since both sulphates and sulphonates are strong acids, (pKa˜1.0) the lower limit of the pH range is around pH 1.0, while the upper limit is provided by the members of the guanidinium group (pKa>13.0). The aliphatic amines (pKa 9-11) are relatively weak bases and the pKa values of the aromatic amines (anilides) are around 4-5 only. Due to the hydrogen bond, the temperature dependence of the system is highly significant. A temperature above the Kraft point should be chosen for micelle formation (in the case of the SDS it is 16° C.). The concentration range is very wide, its lower value is determined by the cmc value of the surfactant applied (in the case of the SDS it is 8.2 mM), while the upper value is limited by the solubility. Obviously, the type of the complexing agent is determined by the concentration of the surfactant. It is reasonable to choose a value exceeding the equimolar value, although depending on the complexing agent, even the partially complexed micelles can be coacervated.

The polymolecular complex coacervation of the surfactant micelles is considered to be significant mainly from the theoretical point of view. In contrast to the conventional complex coacervation applying primarily natural substances, in this case the composition of the system is accurately known, the surface is chemically homogenous, the shape is regular, all the parameters can be measured and calculated. In almost every case the coacervates were water clear, homogenous and they did not contain inclusions. The micelle-crystals—fixed metastable colloidal systems which could be studied easily, —are well utilizable in studies on micelles and colloidal systems. The micelle-crystals can be vesical-like and they should either have a double wall or the their wall is composed of micelle units, or they are made up of the aggregation of smaller micelles. The coacervation of the alkyl sulphate esters and guanidinium compounds can be associated with the origin of life.

The most important practical application of polymolecular complex coacervation is the coacervation of associates prepared by the direct or indirect reaction of surfactant micelles with other molecules. In the associates, the characteristics of the surfactant micelles determine the characteristics of the associate, although the attachment of other molecules has an impact on these properties. We might as well say that coacervating surfactant micelles take, carry or maybe “pack” their associates. The hydrophobic portion of the anionic surfactants primarily interacts with lipophylic molecules and the interaction can be strengthened by the positive charge of the surfactant (e.g. certain proteins, oils and synthetic polymers). Indirect interaction can be e.g. a stain, radioactive isotope or drug dissolved in a lypophilic substance, or in a different system a carbohydrate or stain associated with a protein. The surfactant molecules interact primarily with oil or proteins, while the dissolved or attached molecules get in contact with the surfactants indirectly.

As a matter of fact, in the process of polymolecular complex coacervation a self-assembling colloid agglomerate of surfactant molecules, complexing molecules and associates is formed. Since the system is self-assembling, from the point of view of coacervation the order of adding the different components is insignificant. Since the system is a self-assembling system, the methods cannot be “improperly performed”. If, for example, the amounts of the components are not correctly added and, therefore, no coacervation is formed, even in this case the method can be completed successfully merely by supplementing the missing component. The correctly selected quality and quantity of the complexing agent provide a smooth interaction between the surfactant and the associate. Thus, the interaction between the surfactant and the associate can be formed both before and after the complexation of the surfactant molecules.

The above holds true for the equilibrium state, however, since it may take a lot of time for the equilibrium to be reached, for practical application it is feasible to add the components in a way that the pre-equilibrial state should be favourable for performing the task. In the system the three main participants are the surfactant, the complexing agent and the associate. Each one of them can interact with one another, which makes the phenomenon very complex. Although it is easy to set up theories, but experimentation is the safest way.

The interaction between the surfactant and the lypophilic substances is the simpler. The lypophilic substances interact with the apolar portion of the surfactant that is why we may handle their associates as “blown” micelles.

The interaction between surfactant micelles and the proteins is more complex. The interaction between proteins and surfactant micelles is very similar to that between complexing agents and surfactant micelles, i.e. in fact proteins also form complexes. However, there is an important difference: a protein can form several interactions identical with the ones of the complexing agents, and this adding up of interactions ensures advantage over the complexing agent. This is why proteins can be involved in coacervation. When the proteins and the surfactant get in contact at first, the complexing agent can occupy the remaining sites, and vice versa, if the surfactant gets in contact with the complexing agent at first, proteins should “compete” for the binding sites. Both cases lead to an equilibrium state, so the question of strategy is just a matter of choice.

It is well known that SDS denaturates proteins, i.e. it disrupts the original configuration of the proteins, and it binds to the individual polypeptide chains. The binding depends on the sequence of the protein. The “head” part of the SDS molecules interacts with the positively charged groups, while the apolar part gets attached to the hydrophobic core of the micelles. As a result of these interactions protein molecules get strengthened (especially when the disulphide bridges are reduced to SH groups) and the SDS micelles attach to the polypeptide chain (as the pearls on a chain).

The complex formed by polypeptide chain and SDS micelles represents a new entity and its properties differ from those of the other micelles. Concerning the polymolecular complex coacervation the most important question is the charge. When completely saturated, the mass of the SDS in the SDS-protein complex is about 2.5-times of that of the protein. Thus, the total charge of the SDS-protein complex is mainly determined by the charge of the SDS molecules and the individual properties of the proteins are oppressed. From among the properties of the complex it is the property of the SDS, which represents an overwhelming ratio, which will be a determining factor later on. In the process of polymolecular complex coacervation the positive charge of proteins is neutralized by the SDS, while it is the cationic complexing agent that can get into contact with the free negative charges. This interaction is not as specific as the bond formed with the SDS anion, but it fits well into the environment and is, therefore, energetically favoured. It can be assumed that in the process of coacervation the charge-neutralized micelles approach each other and they fold with the polypeptide chain to form a sphere-like, or in any case, follow the tightest possible packing. In the solution the structure of the complexing agent-SDS protein system can be stabilized further by taking up free SDS molecules or the micelles at large.

Although SDS is especially suitable for this purpose, other anionic surfactants of the invention can also be used to take the proteins into the micelles, provided the surfactants meet the above criteria and can form secondary bonds with the protein in question. Of course, the formation of bonds also depend on the hydrophobicity, charge and the number of complexing groups of the protein. Supposedly, a protein carrying several hydrophobic groups can be taken into the micelle easier, and compared to a hydrophilic group the protein will be rather situated inside the micelle. However, the lysine and arginine side chains will enhance a stronger complexing with the anionic surfactant.

Contrary to the conventional complex coacervation, the polymolecular complex coacervation has an importance not only in producing microcapsules but also in applying methods based on phase separation. These methods have at least the same, or perhaps even greater importance. The reason for this is the fact that phase separation methods allow separation of not only the surfactant micelles themselves but also the materials attached to them from other substances remaining in the equilibrium solution. For example, proteins and nucleic acids constitute the two most important groups of macromolecules of biological systems. It is well known, that the SDS interacts with proteins but not with nucleic acids and this phenomenon provides the possibility of these very important groups of biological substances to be separated by using the polymolecular complex coacervation of the invention.

Thus, one of the applications based on phase separation is obtaining, separating, concentrating and stabilizing substances that directly or indirectly form association complexes with anionic surfactants or micelles composed of them. In a sample containing substance(s) that can form association complex(es) with anionic surfactants or micelles composed of the surfactants, association complex molecules of colloid size are produced by adding suitable amount of surfactants and, if necessary, by accelerating the process by warming. Complex coacervation is effected by appropriately complexing the association colloids. In this process, the association colloid and the substance in question constituting a part of it will be enriched in the coacervate. The substance can be obtained from the coacervate by the removal of the equilibrium solution. For example, provided that the proteins and the lipids were to be obtained from a biological sample, after adding the suitable amount of SDS and then the complexing agent, the system is homogenized. The coacervate will be enriched in proteins and lipids. Due to the density differences the coacervate formed with the lipids will be above, while the coacervate formed with the proteins will lie below the equilibrium solution. Thus, by the isolation of the individual phases, the proteins and the lipids can be obtained separately.

By using the same method, a protein sample can be separated from its disturbing components and the sample can be concentrated at the same time or, for example, an aqueous solution can be made oil-free. In the process possible interactions of the components of the solution with the given surfactant should be taken into account. Accordingly, the substance in question can be freed only from the components interacting with the given surfactant. The formation of coacervation is also a kind of stabilization, since the substance in question will be protected inside the colloidal capsule. The substance can be simply obtained from the coacervate by disrupting the association complex and/or the micelles (such as adding more SDS or organic solvents).

The other preferred application of the invention is the separation of substances that directly or indirectly form association complexes with anionic surfactants or micelles composed of them and the related purification process. The purified micelles may as well be labelled. The units of the coacervate formed from SDS-saturated proteins are composed of the agglomerate of each of the polypeptide chains and the micelles bound to them. Since energetically it is the minimal surface that is the most stable, it is likely that the micelles do not keep their original, individual sphere-like shape during the process of coacervation, but by way of fusion they will so to say pack the proteins. Although the particles containing different proteins have uniform surface, they have different volume, thus they can be ideally separated and purified by gel filtration.

Coacervates that are not too compact and have large volume are the most suitable for isolation, since recovery is easier from them. The presence of crosslinks are especially disadvantageous, since in this case re-solubilization with the detergent is difficult, due to close attachment of micelles and occluded parts thereof. The application of the butylamine/SDS, isobutylamine/SDS or the cyclohexylamine/SDS system is regarded to be the most appropriate for this purpose. Since SDBS and guanidine compounds provide bonds stronger than the ones obtained through the SDS/amine complexing agents system, it is advisable to apply the latter one here.

When SDS is added to proteins in an amount smaller than would be sufficient for saturation, the coacervation will depend on the pH and the individual proteins will coacervate depending on their isoelectric points (pI). The charge of proteins that have pI value higher than the pH of the solution will be neutralized and the protein will coacervate, whereas the other proteins will remain in the equilibrium solution; and the farther is their pI value from the pH of the solution, the more negatively charged they will be. Separation of these proteins may be based either on the phenomenon that coacervation of proteins is pH dependent, or on the fact that at a given pH the charge of proteins may vary from zero to a given maximum.

On the basis of this principle, heterogeneous separation can be performed on suitable solid phase, e.g. a chromatographical separation on a column packed with suitable medium. For example, during the coacervation process the proteins that bind to a suitable ion exchange column will be internalized into the micelles and, since the surface charge of the micelles is neutral, the proteins can be eluted.

A further possibility is the use of a gel filtrating column having an appropriate pore size and the elution can be performed by the aid of a pH gradient. While the small-sized, non-coacervated proteins migrate slowly, those that coacervate at a given pH and, thus, internalized into large-sized micelles will quickly pass through the column.

It is likely that the separation of coacervate composed of different proteins can be performed by hydrophobic chromatography.

When choosing the suitable system, the fact that the isolation is performed in liquid phase should be taken into consideration, thus, the most important criterion is to select such a system that will not solidify at the temperature of separation. For chromatographic separation further important criteria are represented by the dynamics and reaching the equilibrium quickly. The inventors mainly concentrate on testing the agmatin/SDS system. It is possible that isobutylamine/SDS and butylamine/SDS systems are also suitable for chromatography. The SDBS can be “too aggressive” to the proteins and it is likely that due to its strong binding to the proteins, it cannot be easy to get rid of it. Since the protein-surfactant complexes represent new entities, each protein can significantly change the process of coacervation (e.g. shape of the micelles or their adhesion etc. may vary; the proteins may be linear or globular, their isoelectric point maybe low or high, etc.). The system appropriate for a given application should be chosen through trials.

Polymolecular complex coacervation can be used for labelling proteins. Since the individual proteins can bind the SDS in an amount that is 2.5-times bigger than the weight of the protein and one SDS molecule binds one complexing agent, therefore, by choosing a suitable complexing agent (such as aniline chloride), each individual protein molecule carries several, well detectable (e.g. in UV) molecules. The other possibility is the absorption of a well detectable molecule (e.g., lypophilic fluorescent stain) in the surfactant used for the coacervation of the protein.

As has been shown in Example 15, a little amount of reporter molecule (such as a stain) can be inserted without changing the characteristics of the coacervation. For example, the production of arginine derivatives conjugated with carboxyl ion is relatively easy and these compounds excellently coacervate. When they are used in only small quantities, the group “hanging” on them—which otherwise would cause problems of steric origin—does not present difficulties. It should be taken into account that if all of the complexing agents applied are large in size, they may need to compete for the space on the surface of the micelle.

Another preferred application of the embodiment of the invention includes the removal of substances that form association complexes with anionic surfactants or the micelles deriving from them directly or indirectly or by the way of inclusion into micelles, as well as obtaining, isolating, purifying and stabilizing compounds that do not form association complexes, such as the different nucleic acids. In the process of polymolecular complex coacervation, the amount of colloid enriching in the coacervate will diminish in the equilibrium phase. This allows removing substances associating with the surfactant from the original solution, and vice versa, isolating and purifying non-associating substances. Since the biological degradation can be related to the enzymes, the removal of these substances also means the stabilization of the remaining substances.

The main steps of the isolation of nucleic acids are the following: the cell lysis, the disruption of nucleic acid—protein complexes, the inactivation of endogenous nucleases and the separation of nucleic acids from the contaminants. In the first three steps SDS is one of the reagents which has been long and most frequently applied. Since complex coacervation provides a possibility for the separation from the contaminants, the whole procedure can be based on the same ground, and this makes this procedure easier to be carried out. For the inactivation of enzymes degrading RNA, guanidine is the one that is most frequently used compound, however, since guanidine is a good complexing agent, it can also be inserted into the method of the invention. According to inventors' experiments, SDS completely inactivates the RNase A enzyme provided that it is used in a saturation amount and if cleavage of the disulphide bridges of the enzyme is ensured. Although SDS is one of the strongest denaturing agents, a significant drawback of its use was that it could not be easily removed. In the method of the invention, the SDS concentration in the equilibrium solution can be reduced to any desired low level.

In many cases not all nucleic acids should be isolated but only certain types (DNA, RNA), as desired. The unnecessary nucleic acids can be digested with a suitable enzyme, then the enzyme (protein) can be removed from the solution by polymolecular complex coacervation. Although the method contains two steps, it produces a clean product, and since the method of the invention is fast and simple, its use is advantageous. Similarly, the method is well applicable for removing proteinaceous contaminants from a solution (e.g. after enzyme reactions, before electrophoresis, etc.).

Nucleic acids obtainable this way are free of lipids and proteins and have a quality that allows them to be used for several purposes (such as screening). The equilibrium solution of the coacervation contains not only nucleic acids but also other compounds with small molecular weight (e.g. amino acids, nucleotides, metabolites, complexing agent, etc.) from which the nucleic acids can be separated efficiently (e.g. by precipitation with alcohol).

According to a further highly preferable method for separating the nucleic acids, the present phase separation of the micellar colloid solutions itself is used for obtaining nucleic acids from various biological samples. This task is expediently accomplished by a second coacervation step performed in the equilibrium solution of the first coacervation. In this way an especially high-purity nucleic acid can be obtained. Provided the second coacervation is performed with a trivalent or multivalent complexing agent (e.g. spermine) and an anionic detergent (e.g. SDS), the nucleic acids get into the coacervate while all the contaminants will remain in the equilibrium solution. The spermine can condense only nucleic acids larger than a specific size (approximately 100 bp), thus nucleic acids can also be isolated from oligonucleotides (such as PCR primers, end products of RNAse or DNAse digestion, labelled nucleotides, etc.).

This nucleic acid isolation method is described below in details.

Among the complexing agents of the polymolecular complex coacervation of the invention the multivalent complexing agents (i.e. molecules comprising several groups capable of forming complexes) constitute a peculiar subset. Molecules comprising multiple cationic groups can simultaneously complex two different micelles, so they are able to form intermicellar interactions. When a large number of the formed, charge-neutralized, complexed micelles are attached to each other through intermicellar interactions a macroscopic structure, i.e. precipitate, will be formed. The use of compounds having more than one group capable of complexing frequently results in precipitates (see Table 1). It is obvious from the table that the increase in the number of complexing groups correlates to the tendency to form precipatete. Data shown in Table 1 are based on the observation of interactions between the individual complexing agents and surfactants under conditions as simple as possible. These data are basic assessments to characterize and compare individual compositions, and may significantly vary as a function of parameters influencing the interaction. By decreasing the strength of intermicellar interactions, coacervation or even a solution state can be effected in a system that basically gives precipitate.

Polymolecular complex coacervation of the invention is a resultant of secondary chemical interactions. The features of the medium have significant influence on the strength of the secondary chemical interactions. Besides the concentration, the pH, ionic strength, dielectric constant and the temperature of the solution have also major impact on the strength of secondary chemical bonds.

The practical importance of multivalent complexing agents lies in the fact that simultaneously with the complexation of surfactant micelles the agents are capable of forming electrostatic interactions with other substances, as well.

It is well known that trivalent or multivalent cations are capable of inducing the condensation and precipitation of nucleic acids. Precipitation of the nucleic acids with spermine is one of the basic methods of molecular biology. Spermine highly selectively precipitates nucleic acids larger than 100 base pairs from aqueous solutions with low salt concentration. Despite the simplicity and selectivity, the method is not widely used. The main reason for this is that due to the network of intra- and intermolecular bonds formed by spermine molecules, redissolving spermine precipitated nucleic acids, especially those of high molecular weight, is very difficult.

It was an unexpected discovery that under appropriate conditions the coacervate formed from SDS and from some of the complexing agents of the invention, e.g. the trivalent or multivalent amines, can form stable interactions with nucleic acids. Table 1 shows that basically spermine, spermidine or the tetraethylene pentamine form precipitates with SDS. It was recognized that if parameters are carefully selected, especially when the strength of intermicellar electrostatic interactions decreases, instead of precipitation, coacervation takes place between the polyamines and the SDS. Factors directing the process towards coacervation instead of precipitation are the polyamine/SDS ratio, as well as the increase of ionic strength, and the decrease of dielectric constant of the solution. The latter—that was achieved by the addition of small molecular weight alcohols in the present study—is especially effective. By the addition of a certain amount of alcohol, coacervation can be terminated and the system can be brought to solution. By varying the parameters, the polyamine-SDS-water system can be brought into either solution, coacervate or even precipitate state, any of which may be advantageous in a given application.

According to accepted theories, the formation of immobilized ion pairs is necessary for the precipitation of nucleic acids [Wallace, D. M., Precipitation of nucleic acids. Methods Enzymol., 152, 41-48 (1987)]. For the precipitation of nucleic acids at least 90% of the negative charges of the phosphate groups should be neutralized with immobilized ion pairs. In the spermine-SDS coacervate one spermine molecule may complex four SDS molecules, i.e. besides the complexation of the micelle, 0-3 amino groups of each spermine molecule may participate in other interactions. Since one micelle is complexed by several spermine molecule, the complexed micelles act as an associated polyamine. This fact is supported by our observation that the spermidine-SDS coacervate—although less effectively—is also able to interact with nucleic acids just in the same way as the spermine-SDS system does. In the SDS-spermidine coacervate the immobilized spermidine molecule may have at most two free amine groups per molecule, which are not sufficient in themselves to precipitate the nucleic acids, i.e. it is not the individual molecules but the complexed micelle that participates in the interaction with nucleic acids. The complexed micelle behaves here like an associated polyamine bound together with secondary bonds. Experiments with negative results, performed with ethylene diamine and agmatine confirm that the complexing agent interacting with nucleic acids should be at least a trivalent or a multivalent polyamine. The pentavalent tetraethylene pentamine can be used as effectively as the quaternary spermine. (Since only technical grade tetraethylene diamine is available, in methods where analytical purity should be maintained its application is not feasible.)

It was an unexpected discovery that the strength of the SDS-polyamine interaction is several fold higher than that of the interaction of the polyamine-nucleic acid. This means the polyamine molecules bind primarily to the SDS-micelles resulting in a coacervate that is more stable in itself than the coacervate-nucleic acid system. In studies of SDS-spermine-nuclic acid system we observed that by increasing the ionic strength of the medium through the addition of concentrated NaCl to the coacervate, the nucleic acid will get into the equilibrium solution. The process is reversible, since by decreasing the ionic strength the nucleic acid can be brought back to the coacervate from the equilibrium solution. The process has a hysteresis, the transition of the nucleic acid from the coacervate to the equilibrium solution and vice versa takes place at different salt concentrations. At ambient temperature and by using non buffered solutions of the components, this transition takes place at a NaCl concentration of 200-300 mM. By increasing the salt concentration up to the 2000 mM, the phenomenon of coacervation still occurs. We concluded that the bonds between the coacervate and the nucleic acid would be disrupted earlier than the bonds between SDS and spermine, which holds the coacervate together.

The system will be more complex if also the dielectric constant of the medium is changed. By the addition of organic solvents, expediently small molecular weight alcohols, the dielectric constant of the system can be significantly decreased. We recognized that by decreasing the dielectric constant of the medium, i.e. —for example—by adding alcohol in an increasing amount, the system can be transferred from the state of precipitate into coacervate and then even into solution state. The process to reach the solution state is significantly affected by the temperature and the salt concentration. At higher temperatures and at higher salt concentration less alcohol is sufficient to initiate the transition from coacervate to solution. Since these are equilibrium processes, the phenomenon is reversible, i.e. by changing the parameters oppositely, the solution can be brought back to coacervate or precipitate state. The phenomenon was explained as follows: the micelles are not real polyelectrolites but associates, thus the organic solvents act not only on the complexation taking place on the surface of the micelles but inside the micelles they weaken the apolar interaction holding the micelles together. As a contrast, the nucleic acids are de facto polyelectrolites, thus the decrease in the dielectric constant of the medium increases the formation of ionic pairs in the nucleic acids, which leads to the condensation or precipitation of the nucleic acids. The rate of alcohol concentration necessary for precipitating the nucleic acids also depends on other parameters, such as temperature, salt concentration, and pH value. It is favorable that the alcohol concentration necessary to precipitate the nucleic acids is higher than the level necessary for the SDS-spermine-nucleic acid system to reach the coacervate or solution state, and this provides an opportunity to recover the nucleic acids in pure form. The state where the complexed micelles bind or unbind the nucleic acids, or where the system is in liquid or coacervate, precipitate may be reached by changing the SDS-sperimine ratio as well as the parameters affecting the strength of secondary chemical bonds. This means that the system may preferably be used for the selective and effective separation of nucleic acids, because by changing the system parameters the nucleic acids can be brought to either the coacervate or the equilibrium phase.

For isolating nucleic acids, from a number of possible strategies we have chosen the one where the spermine-SDS system is added in a solution form to an aqueous solution of the nucleic acid. The solution state was ensured by the addition of a suitable amount of salt and alcohol. The alcohol and salt concentration of the solution state spermine-SDS system decreases during the addition to the nucleic acid containing sample. As a result, the solution-state spermine-SDS system will coacervate and it interacts with the nucleic acid. When the equilibrium has been reached, the nucleic acid-spermine-SDS coacervate gets separated from the equilibrium solution and thereby from all the contaminants. In the subsequent step homogenous solution is formed from the separated coacervate by adding an appropriate amount of salt and alcohol. Then the alcohol concentration of the solution is decreased while the salt concentration is maintained at a high level by the addition of a suitable amount of salt to the homogenous solution. If needed, the pH of the solution is preferably increased and its temperature is decreased. If the parameters were set this way, the system became a coacervate again, but due to the high salt concentration the nucleic acid did not get into the coacervate but remained in the equilibrium solution. Finally, pure nucleic acid free of contaminants can be recovered by separating it from the coacervate.

As compared with the spermine precipitation of nucleic acids the use of SDS-spermine coacervate offers several advantages. While the spermine treatment leads to the separation of nucleic acids in a shrinked precipitate netted with the intra- and intermolecular bonds of the spermine, by using the method of the invention the nucleic acids can be recovered in a form embedded into colloidal-sized, fluid micelles. The SDS-spermine coacervate interacting with the nucleic acids is an associated polycation, i.e. a system comprising small molecules which—both in terms of geometry and charge distribution—can follow the structure of the polyanion involved in the interaction with the highest possible flexibility. The colloidal size of the coacervate provides a possibility for nucleic acids to immobilize in the absence of strain and without intermolecular bonds. The electrostatically bound nucleic acid molecules that are embedded in high viscosity and flexible coacervate are protected against biological, physical and chemical degradation, thus the SDS-spermine coacervate is a suitable medium for transporting and storing nucleic acids. The recovery of nucleic acids—even the large, genomial DNAs—from the coacervate is a simple and quick procedure.

By using the method of the invention even the trace amounts of nucleic acids can be isolated effectively since they can be separated in an arbitrary amount of coacervate. The coacervate as “carrier” quasi collects the nucleic acid molecules providing their effective separation. In the method of spermine precipitation it is a frequently occurring event that although a portion of the nucleic acids is condensed but cannot be sedimented even by using high speed centrifugation.

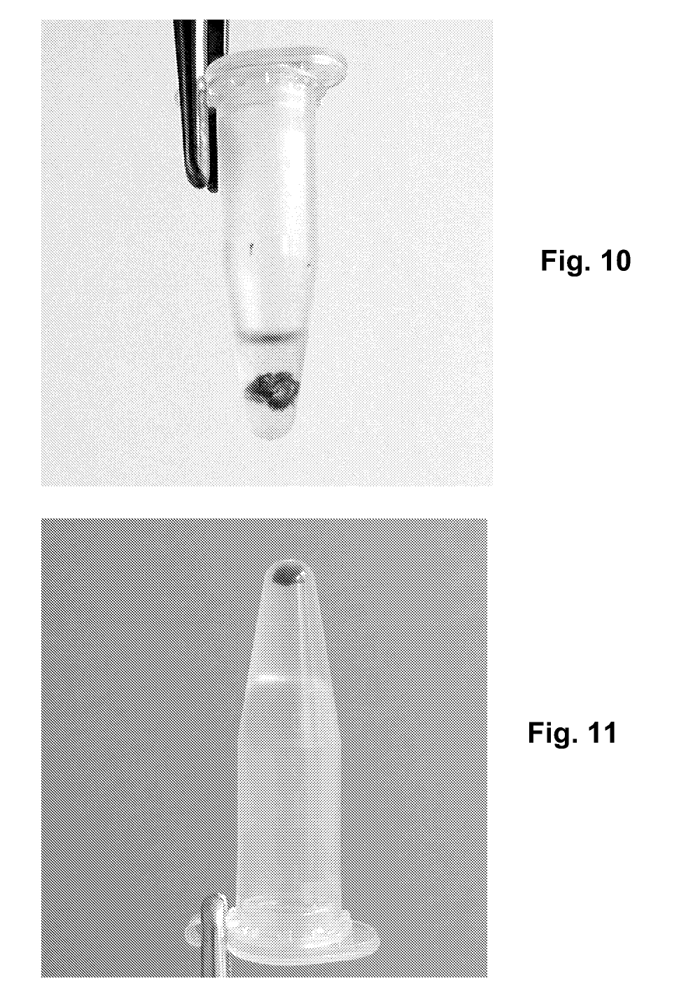

A further advantage of the invention is that by the addition of certain stains or dyes, the coacervate, thus the nucleic acid to be isolated, can be made visible (FIG. 10). Surprisingly, protein stains or dyes, such as Brillant Blue R-250 (CBB) dye used in other examples of the invention, can be used for visualization. In these applications the protein stains or dyes (Ponceau S, Naphthol Blue Black, Brillant Blue) do not actually stain the nucleic acids but by forming complexes with spermine they can be built in the coacervate and thus indirectly label the nucleic acids. The visualization of nucleic acids will effectively help to reduce the loss.

By dispersing SDS-spermine-nucleic acid coacervate built of colloidal units, nucleic acid containing microcapsules can be produced. Uncharged, lipophilic, nucleic acid containing capsules consisting of associated small molecules can be ideal vectors for transfecting living cells.

The SDS-spermine system would well fit to the protein- and lipid-coacervating system of the invention. The protein and lipid free equilibrium solution of the latter may serve as a starting material of the SDS-spermine system. Since in the first step, the nucleic acids will undergo a negative selection while, in the second step, a highly selective positive selection, extremely high purity nucleic acids can be recovered even from the most complex biological samples.

The method of the invention comprises remarkably simple steps, and no significant physical forces that may break down the nucleic acids occur during the process. The polymolecular complex coacervation is based on the application of secondary chemical bonds. Contrary to real polycations constructed by covalent bonds, the coacervate, an associated polycation, interacting with the nucleic acids will bind the nucleic acids through electrostatic interactions. Due to the above features, intact nucleic acids can be obtained by using the method. Unlike other well-known methods, the method of the invention is particularly preferably applicable for the isolation of high molecular weight genomial DNA.

Basically, the phenomenon of the polymolecular complex coacervation provides an opportunity to separate the detergent and the complexing agents by separating the coacervate containing higher density colloid particles from the equilibrium phase which contains the components in very low concentration. The practical importance of the polymolecular complex coacervation lies in the fact that the components comprising coacervate can interact not only with each other but also with other substances. Due to this secondary interaction, these substances may become the constituents of the neutralized colloid particles, i.e. the coacervate, thus they will be saparated from substances not participating in the interaction. The secondary interactions of both the surfactants and the complexing agents are preferably used in colloid phase separation. By using the polymolecular coacervation of the invention the phase separation of the most important biologically active substances can be carried out. The detergents can interact with lipids, oils and proteins, and among the complexing agents the trivalent or multivalent polyamines can interact with nucleic acids in such a way that these biomolecules will become parts of the uncharged colloid particles, i.e. the coacervate. The practical utility of the polymolecular coacervation can be summarized as follows:

The coacervate can be separated from the equilibrium phase, thus substances either involved or not involved in the interaction can be separated.

The coacervate, i.e. the phase rich is colloid particles, can be dispergated into its elements, providing microcapsules of the separated substances.

The substances enclosed in the coacervate are protected against physical, chemical and biological degradation, thus the separated and coacervate-enclosed substances can be stored and transferred.

By disrupting the equilibrium state of the coacervate, i.e. small molecules which are held together by secondary chemical bonds, the substances enclosed in the coacervate can be recovered.

Any application of the invention that includes the removal of the contaminants is mainly aimed at the equilibrium solution. According to the law of mass action, the equilibrium solution always contains micelles, however, the amount of the micelles can be reduced to a desirably low level (see example 6). The application of well coacervating systems is advisable, thus—besides the emulsion-formers—all such systems can be used appropriately. In the practice of the invention it is advisable to use a system that coacervates at ambient temperature, since the warming-cooling process represents a separate technological step that unnecessarily increases the number of the steps in the method. Well applicable systems are e.g. agmatine/SDS, butylamine/SDS or SBDS, isobutylamine/SDS or SBDS, and cyclohexylamine/SDS.

Since density of the coacervates formed by butylamine or isobutylamine barely differs from that of the water, the two phases do not separate distinctly. Upon warming, the coacervate will rise and it will form an upper phase, however, at certain temperatures it will form a sphere in the middle of the equilibrium solution. These phenomena can be advantageous in certain applications. Surprisingly, when NaCl is added to the solution (wherein the definite rising of the coacervate is expected) just the opposite phenomenon can be observed. When the salt concentration is raised, the volume of the coacervate decreases while its density increases, and the coacervate definitely forms a lower phase. In the case of the above-mentioned amines, the volume of the coacervates is significant (about 40%). This means, that the substance remaining in the equilibrium solution will get concentrated. In a different embodiment the more compact the coacervate is the better; the aim here is the use of a system getting cleaner ever faster and the provision of an even cleaner equilibrium phase. This can be achieved by the use of the agmatin/SDS system having several functional groups. The increased number of functional groups (the strong guanidine and the weaker amine groups) ensures that not a precipitate but a compact coacervate phase is formed. The ratio of the coacervate is only about 20% in this system, which becomes clear in minutes. A similar result can be achieved when a mixture of complexing agents is used. If bifunctional complexing agents (such as spermidine, hexamethylene pentamine) are added to the above amine-type complexing agent, the compactness of the coacervate can be increased. The suitable ratio can be set only experimentally, since already a small change in the ratio can cause significant change in the characteristics of the coacervate.

There would be certain cases when the choice of coacervation performed at higher temperature is rewarded by a reliable and very efficient separation of the solidifying coacervate and the equilibrium solution. The fact that phase separation can be performed in automatized systems in a simple and safe way may justify this solution. The application of the guanidine/SDS system could have the following additional advantage: after the coacervation performed in great excess of guanidine, the equilibrium solution contains the chaotropic substance (in the example it is the guanidine) necessary for the silicate based adsorption purification of nucleic acids, in a sufficient amount. Therefore, the solution can be directly used for this purpose.

The further preferred application of the polymolecular complex coacervation of the invention is the production of microcapsules. Since the size of the micelles is in the submicron range, nanocapsules can also be produced. The size of the capsules depends on the size of the micelles and the degree of their association. Microcapsular inclusion of substances associated directly or indirectly with anionic surfactants can be performed by the polymolecular complex coacervation of the association colloid system. The use of systems showing micelle-crystallization at the temperature above ambient temperature is particularly preferred, since the cooling of the warm coacervate will result in a product covered with a solid shell which can be used without any post-treatment. Of course, there is a possibility to perform the conventional wall-strengthening method based on crosslinks, however, in this case the complexing agent should provide the functional groups suitable for crosslinking on the surface of the wall.

At ambient temperatures, micro- and nano-emulsions can be produced by the dispersion of the coacervate. In this case, those complexing agents are preferred which provide such a surface for the association colloid, which blocks or regulates the attachment of the elemental units. Here the association colloid consists of complexed anionic surfactants, surfactant micelles and substances associating directly or indirectly with the micelles. As a matter of course, it is also possible to apply supplements inhibiting or regulating the attachment.

When a suitable surfactant—complexing agent pair is selected for the production of micro- and nano-capsules, the most important factor to be considered is the creation of a wall of suitable strength. Micro- or nanodispersions, or micro- or nanocapsules are related terms with largely overlapping meaning, as they are (or comprise) solid particles in each case. For these types of application it is preferred to use systems coacervating above ambient temperature (or above the temperature of application). The guanidine/SDS or SDBS, aniline/SDS or SDBS, ethylenediamine/SDS, cyclohexylamine/SDBS, arginine/SDBS systems are considered to be well applicable. An especially important system is represented by the hydrazine/SDS system which coacervates at 30° C., i.e. at a temperature range near to room temperature. The capsule that is solid at room temperature becomes “liquid” at body temperature. The content of the capsule should be considered at the selection of the applied complexing agents. For packaging oily substances it is not an appropriate choice to select systems which are based mainly on hydrophobic interaction. The aniline/SDS system is suitable for the proteins, but it does not retain oils well enough. For packaging oily substances the guanidine-type, i.e. a system with strong hydrogen interaction, is preferred (e.g. guanidine/SDS, arginine/SDS). It is also preferable when the coacervate is not too compact, so that the individual particles may remain separated during cooling and micelle-crystallization. The applied parameters (surfactant/complexing agent ratio, surfactant concentration, etc.) affect the characteristics of the coacervate, the shape of the coacervation; thus, a person skilled in the art can optimize the process.

The micro- and nanoemulsions themselves are suitable for several purposes, such as for solubilizing and targeting different active ingredients, or they can be used in detergents, stains or dyes and adhesives. For the production of micro- and nanoemulsions the best systems are those where coacervation is not very pronounced, i.e. the hydrate shell becomes only thinner, thus, the attachment of the colloid particles will stop when a given particle size is reached. The SDBS/arginine, SDBS/hydroxylamine, SDBS/hydrazine systems serve this purpose well. The formation of the complex can take place in the aqueous solution of polyvinyl alcohol (or glycerol).

As a conclusion, when selecting the complexing agent(s) to be used it is advisable to consider the objective of the method. In the production of microcapsules those complexing agents are preferred which coacervate at higher temperature and form micelle-crystals at ambient temperature. To obtain associates, complexing agents from which associates can easily be recovered are to be used. When the task is to remove associates, those systems are preferred that result in quick and efficient association of the elemental units of the coacervate. Thereby, a fast clarification of the equilibrium solution can be achieved. Furthermore, the coacervate to be removed can be obtained in the form of compact precipitate on the bottom of the flask and the solution containing the nucleic acid can be easily decanted.

The micelles contained in the systems according to the invention can be studied by any method known in the art for studying micelles. An especially preferred method is the Small Angle Neutron Scattering, SANS (see 5, 6, 7). Other preferred methods are the Electron Paramagnetic Resonance (see 7), the Spin-Probe Study (see 8) and the Time-resolved Fluorescence Quenching Measurements (see 9) techniques. Furthermore, various high performance light- and electronmicroscopical techniques can be used as well. For the purpose of studying associations the Biachore instruments are well applicable.

### EXAMPLES

The following examples are aimed at illustrating certain embodiments of the invention and they are not intended to limit the scope of the invention. The purpose of these examples is not to elaborate the condition of the optimized applications; they merely seek to illustrate the applicability, the possibilities and the limits of polymolecular complex coacervation.

### Example 1

### Interactions of Surfactants and Complexing Agents

Hundred microliters of surfactant solution (0.5 M) were measured to a 1.2-ml reagent tube (To decrease the viscosity 200 μl of water was added to each of the SDBS solutions), then 100-100 μl of each of the surfactant solutions (1 M) were measured to the surfactant and the solution was vortexed. (To provide a protonated state that is important for all the complexing agents, the solutions of the complexing agents were used at neutral pH, while in the case of the anilides the pH was mildly acidic). If precipitation occurred, the centrifuge tubes were warmed up stepwise (in 5° C. steps), optionally, up to 95° C. In a few minutes, the samples were checked macroscopically and microscopically. The results are shown in Table 1 below. Due to the special application possibilities, among the agents providing coacervation the emulsion forming capability of complexing agents was expressly indicated. The term “viscous solution” in the table means that the complexing agent cannot reduce the hydrate shell surrounding the micelles to a level necessary for forming a separate phase. The indication “precipitate” means that a precipitate or agglomerate of micelles fixed in a network were formed due to the interaction. The label “coacervate” means that the complexing agent does not tend to form micelle-crystals. It should be noted, that every system has a tendency to “overcooling”; i.e. the temperature where micelle-crystallization is initiated can vary significantly. The results are shown in Table 1. In practice, the complexing agents listed in the table were added in the form of chloride or sulphate salts.

On the basis of the results it can be concluded that a wide range of compounds which contain ammonium or guanidinium groups can be used, and due the complex nature of the complexing process, the range of suitable compounds cannot be definitely limited. The results suggest that ionic bonds and further secondary bonds have synergistic effect. Secondary bonds bring the cation close to the anion, which results in the strengthening of the electrostatic interaction (which changes by the square of the distance). Finally, the cation gets immobilized close to the anion and by occupying its parts capable of forming bonds, it displaces the original counterion (Na⁺) as well as the water molecules that can form only simple (i.e. not complexing) bonds.

It is apparent that while the ethyl- and propylammonium-chloride does not coacervate with SDS, the butyl- and other ammonium salts having longer carbon chain can form a coacervate therewith. We can infer the role of the hydrophobic interaction from this observation.

In the case of hydrazine chloride the necessary additional interaction is provided by the doubling of the hydrogen bonds and the ionic bonds. The hydrazines, which might be regarded as a special variation of the amines, are also supposed to be good complexing agents. The ionic-dipol, dipol-dipol, the induced ionic-dipol, etc. bonds are formed in almost every structure and these bonds strengthen the simple ionic bond.

Besides the compounds listed in the table, there are several other compounds that can form complexes. If we just only check the homologous series, there are several complexing agents that are worthy of note. The inventors do not see any theoretical obstacles to the enablement of the invention, the basis of which is the phenomenon of the new type of colloid phase separation, by using any anionic surfactant—cationic complexing agent pair that can form ionic interaction together with further secondary interactions, preferably at least a hydrogen bond. It is obvious that it is necessary to find the suitable conditions for this and these conditions may significantly differ from those applied in the present example, which can be easily set up in a laboratory. Thus, it can be easily imagined that the compound-pairs of the table that could not initiate coacervation or emulsification under laboratory circumstances applied, can be used for the phase separation of colloid systems in suitable circumstances.

The nitro-guanidine shown in the last row of the table was tested for the purpose of verifying the importance of the cationic property. The nitro-guanidine—due to the electron-withdrawing property of the nitro group—cannot be protonated; it does not form a guanidinium group, thus, it is not surprising that it does not coacervate. By summing up the results, suitable cations can be the cations of general formula I, R,R′,R″NH⁺(where R means hydroxyl or substituted or unsubstituted alcoxy, aryloxy, amino, alkyl, alkenyl, alkynyl or aryl group, or it can be an amine or hydroxy group, R′ and R″ means H or substituted or unsubstituted alkyl, alkenyl, alkynyl or aryl group, R′ and R″ can be identical or different) and the ones of general formula II, R—NH—C⁺(NH2)-NH—R′ (where R and R′ means H or substituted or unsubstituted alkyl, alkenyl, alkynyl or aryl group, R′ and R″ can be identical or different).

| Complexing agent | General | |
|---|---|---|
| (unprotonated form) | formula | R |
| 2-guanidino-benzimidazole | II | 2-benzimidazole |
| acetyl-guanidine | II | —O(O═)C—CH₃ |
| agmatin | II | —(CH₂)₄—NH₂ |
| aniline | I | —(C₆H₅) |
| arkain | II | —(CH₂)₄—NH—C(═NH)NH₂ |
| arginine | II | —(CH₂)₃—CH(NH₂)—C(═O)OH |
| arginine ethyl-ester | II | —(CH₂)₃—CH(NH₂)—C(═O)O—CH₂—CH₃ |
| butyl-amine | I | —(CH₂)₃—CH₃ |
| Cyclo-hexyl-amine | I | —C₆H₁₀—NH₂ |
| 1,6-diamino-hexane | I | —(CH₂)₆—NH₂ |
| 2-diethylamino-ethylamine | I | —(CH₂)₂—N—(CH₂—CH₃)₂ |
| ethanol-amine | I | —CH₂—CH₂—OH |
| ethyl-amine | I | —CH₂—CH₃ |
| ethylene-diamine | I | —(CH₂)₂—NH₂ |
| 2,2′-(ethylenendioxy)- | I | —(CH₂)₂—O—(CH₂)₂—O—(CH₂)₂—NH₂ |
| diethylamine | | |
| guanidine | II | —H |
| hydrazine | I | —NH₂ |
| hydroxylamine | I | —OH |
| isobutylamine | I | —(CH₂)—CH(CH₃)₂ |
| chlorhexidine | II | —C(═NH)—NH—(CH₂)₆—NH—C(═NH)—NH—C(═NH)—NH—(C₆H₄)—Cl |
| N-methyl-aniline | I | —(C₆H₅) |
| spermine | I | —(CH2)3-NH—(CH2)4-NH—(CH2)3-NH2 |
| spermidine | I | —(CH₂)₃—NH—(CH₂)₄—NH₂ |
| tetraethylene-pentamine | I | —(CH₂)₂—NH—(CH₂)₂—NH—(CH₂)₂—NH—(CH₂)₂—NH₂ |
| triethyl-amine | I | —CH₂—CH₃ |
| tris(hidroxymethyl)amino-methane | I | —C—(CH2-OH)₃ |
| tris(2-amino-ethyl)amine | I | —CH₂—CH₂—N(—(CH₂)₂—NH₂)₂ |
| guanidine + butylamine (1:1) | | |
| guanidine + 4% Triton X-100 | | |
| Cyclo-hexil-amine + 1% Sarcozil | | |
| Nitro-guanidine | 0 | — |

| Complexing agent | | | | | Brillant |
|---|---|---|---|---|---|
| (unprotonated form) | R′ | R″ | SDS | SDBS | Blue R-250 |
| 2-guanidino- | H | — | coacervates | emulsion | |
| benzimidazole | | | (95° C.) | | |
| acetyl-guanidine | H | — | solution | coacervates | |
| agmatin | H | — | coacervates | precipitate | precipitate |
| aniline | H | H | coacervates | coacervates | precipitate |
| | | | (60° C.) | (70° C.) | |
| arkain | H | — | precipitate | precipitate | |
| arginine | H | — | solution | emulsion | |
| arginine | H | — | viscous | coacervates | |
| ethyl-ester | | | solution | | |
| butyl-amine | H | H | coacervates | coacervates | coacervates |
| Cyclo-hexyl- | H | H | coacervates | coacervates | |
| amine | | | | (40° C.) | |
| 1,6-diamino- | H | H | viscous | precipitate | |
| hexane | | | solution | | |
| 2-diethylamino- | H | H | viscous | precipitate | |
| ethylamine | | | solution | | |
| ethanol-amine | H | H | solution | coacervates | |
| ethyl-amine | H | H | viscous | emulsion | |
| | | | solution | | |
| ethylene-diamine | H | H | coacervates | precipitate | |
| | | | (60° C.) | | |
| 2,2′- | H | H | precipitate | emulsion | |
| (ethylenendioxy)- | | | | | |
| diethylamine | | | | | |
| guanidine | H | — | coacervates | coacervates | precipitate |
| | | | (55° C.) | (95° C.) | |
| hydrazine | H | H | coacervates | emulsion | |
| | | | (30° C.) | | |
| hydroxylamine | H | H | solution | emulsion | |
| isobutylamine | H | H | coacervates | coacervates | |
| chlorhexidine | —(C₆H₄)—Cl | — | precipitate | precipitate | |
| N-methyl-aniline | —CH₃ | H | coacervates | precipitate | |
| spermine | H | H | precipitate | precipitate | |
| spermidine | H | H | precipitate | precipitate | |
| tetraethylene- | H | H | precipitate | precipitate | |
| pentamine | | | | | |
| triethyl-amine | —CH₂—CH₃ | —CH₂—CH₃ | solution | coacervates | |
| tris(hidroxymethyl)amino- | H | H | solution | solution | |
| methane | | | | | |
| tris(2-amino-ethyl)amine | H | H | precipitate | precipitate | |
| guanidine + | | | coacervates | coacervates | |
| butylamine (1:1) | | | | | |
| guanidine + | | | coacervates | coacervates | |
| 4% Triton X-100 | | | (45° C.) | | |
| Cyclo-hexil-amine + | | | coacervates | coacervates | |
| 1% Sarcozil | | | | | |
| Nitro-guanidine | — | — | solution | solution | |

### Example 2

### Coacervation Depends on the Temperature

It is obvious from Table 1 that the coacervation between certain surfactants and certain complexing agents takes place at a temperature higher than room temperature. Under a certain temperature the complexed micelles become solid, the metastable state becomes fixed. When the system has been warmed again and has reached a critical temperature, the solid material in it will “melt”. To prove that the phenomenon is not dissolution but melting (analogous to the melting of nucleic acids) the following study was performed. SDS (100 μl, 0.5 M) was added into two 1.2-ml reaction flasks. Guanidinium chloride (1 M, 300 μl to one sample and 550 μl to the other) and water (150 μl) was added to the samples, thus, while the concentration of the excess guanidinium-chloride remains unchanged, the volume of the first solution is half of the second. Both flasks were warmed to 60° C. then quickly cooled down. Flasks containing the white precipitate were warmed up to 50° C., then a white precipitate could be found on the bottom of both flasks in equal amounts. When the temperature was raised to 55° C. in the two flasks, the precipitate disappeared and coacervate with the volume equal with that of the previous white precipitate appeared. This phenomenon cannot be explained by dissolution, because a double amount of solvent would result in a double amount of material. The inventors think that hydrogen bonds compaginating the solid material will disrupt at 55° C., therefore the phenomenon is, actually, a melting and not a dissolution.

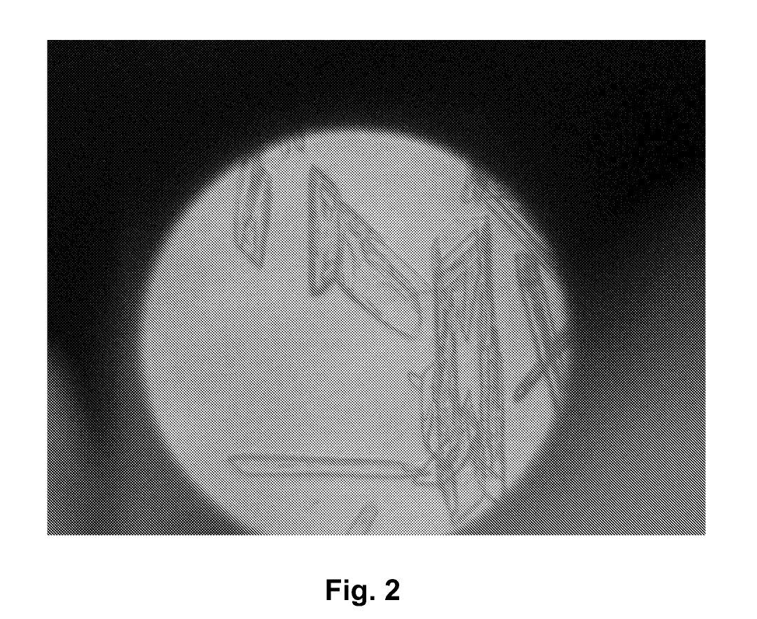

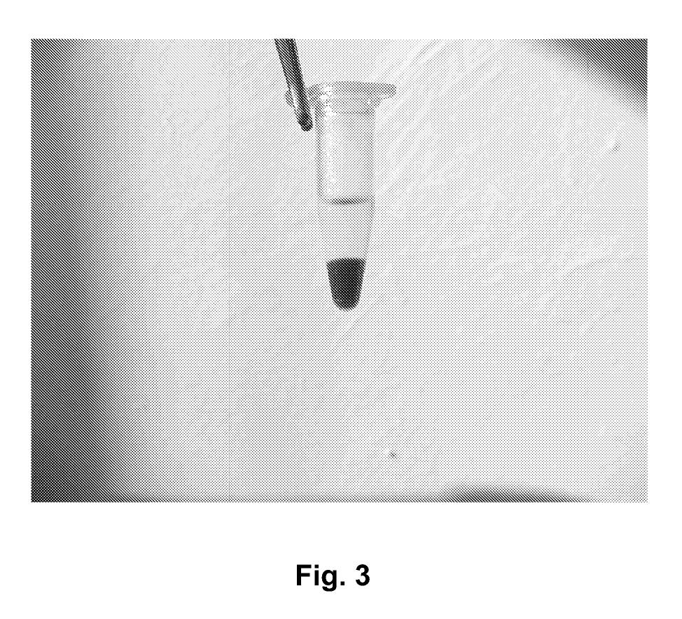

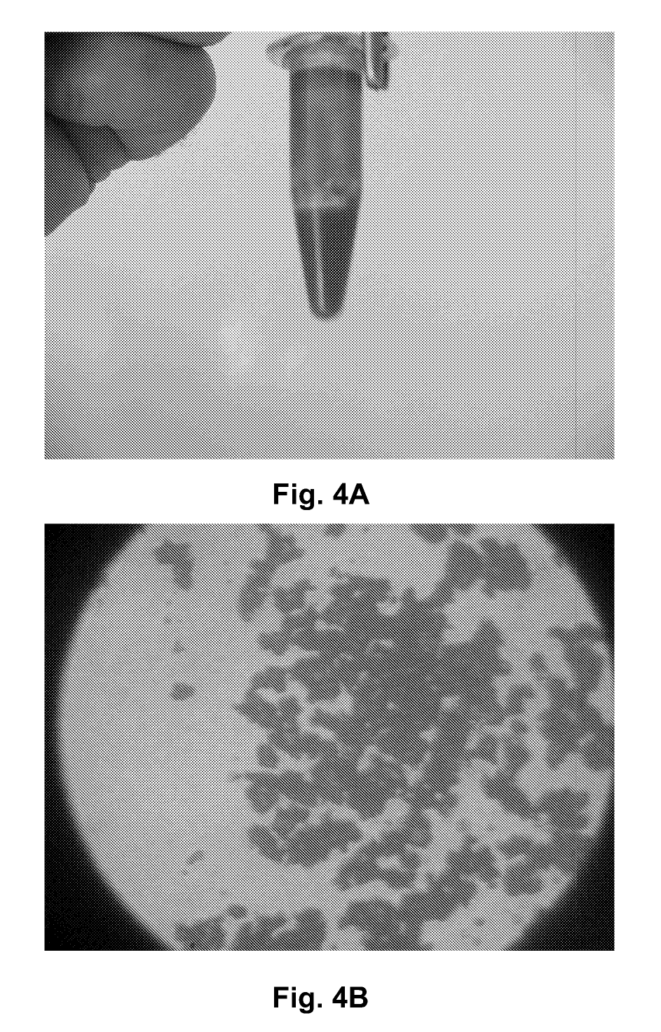

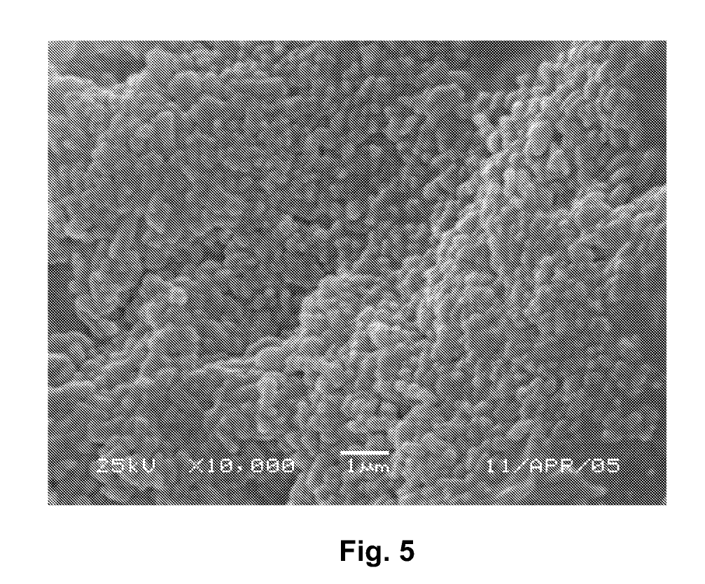

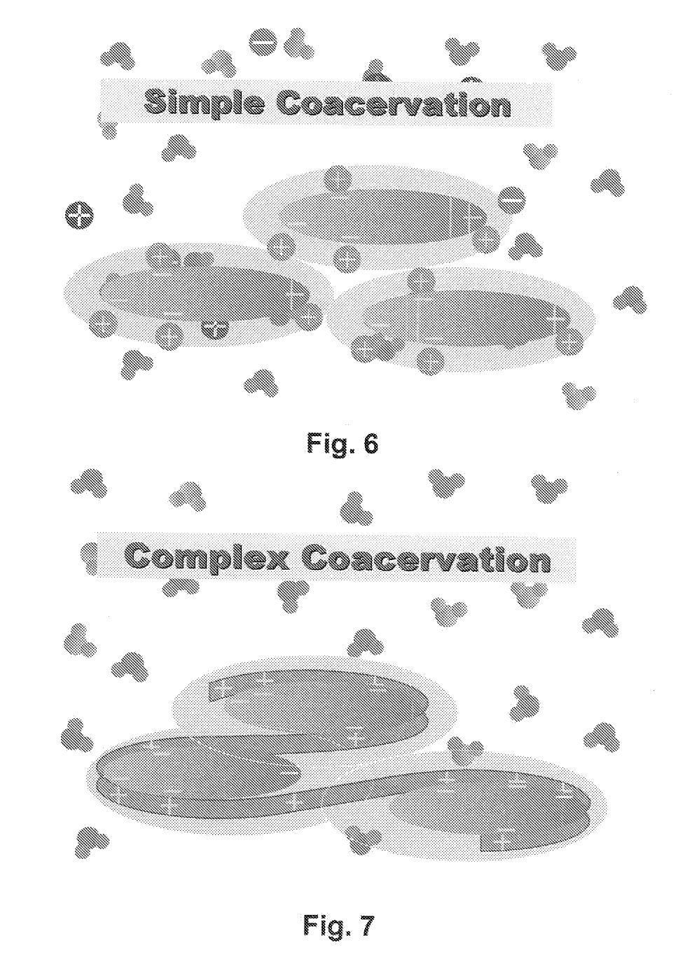

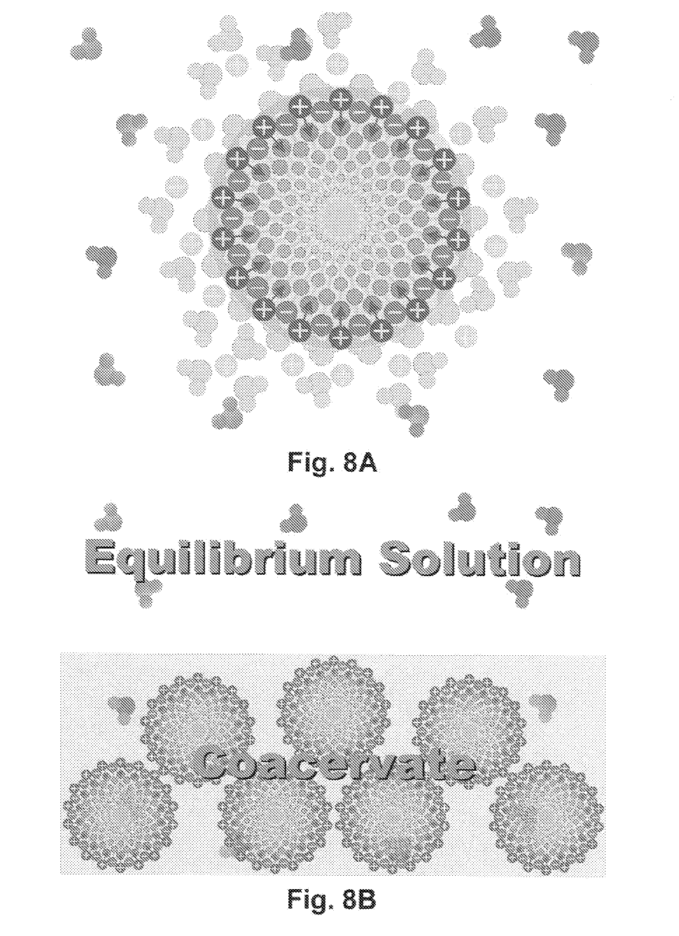

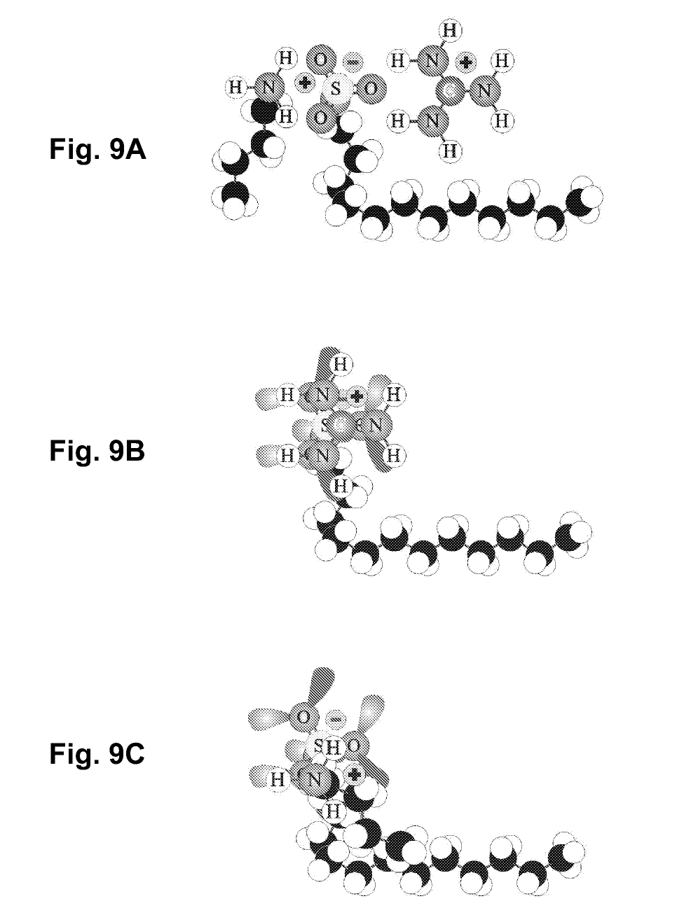

To demonstrate the difference between the micelle-crystalline state formed through coacervation and the regular crystalline state resulting from precipitation (see FIGS. 1 and 2) the following study was performed. Identical compounds were added into two 1.2-ml reaction flasks: SDS solution (100 μl, 0.5 M) and Brilliant Blue protein stain solution (20 μl, 0.1%) were mixed with BSA solution (50 μl, 100 mg/ml). Guanidinium chloride (100 μl, 1 M) was added to the homogenized mixture, then it was vortexed and the first reaction flask was centrifuged (3 min, 10000 g). After centrifugation the system got separated into blue precipitate and blue supernatant. The presence of the blue colour in both phases showed the presence of protein in both phases. According to the microscopical examination, the precipitation was composed of inhomogeneous, regular crystals. The second reaction flask was warmed to 60° C., vortexed and incubated for 5 min at 60° C., which resulted in the coacervation of the solution. The mixture separated into a water clear equilibrium solution and a deep blue coacervate (see FIG. 3). (In a repeated experiment, the UV spectrophotometry proved that the equilibrium solution was free of protein (which coincides with the result of the protein-stain study). The reaction flask was vortexed and cooled during the process. In a certain time the solution faded, then a solid, silky blue, slowly precipitating, but filterable dispersion was formed (FIG. 4). The microscopical examination displayed billions of particles with the size of around a micrometer (FIGS. 5 and 6).

The results are assessed as follows, and this assessment is not intended to limit the invention to any extent.

The characteristics of the complexed micelles are determined by their components, i.e. the surfactants and the complexing agents. Thus—besides the common characteristics—every system has individual properties. In every case the rate of hydration in the complexed micelles is different. The upper and lower limits represent the solution and precipitation forms, which correspond to the complete hydration and the dehydrated form, respectively.

When the hydration rate decreases, the first stage of this process is a viscous solution. The inventors think that in this state the hydrate shell is still significant, but it gets immobilized around the colloid particles to some extent, resulting in an increase of viscosity. The next stage is the emulsion. According to the invention this is already considered to be a phase separation. The extent of the hydrate shell gets significantly reduced and limited interactions develop between the colloid particles. Although, microscopical aggregates are formed, the process gets terminated at this level or it just very slowly continues towards the macroscopically observable separation of the phases. The further reduction of the hydrate shell results in a higher rate of aggregation, which manifests itself in the macroscopical separation of the phases, i.e. the development of a coacervate. [The first sign of coacervation is always that the solution becomes turbid then the aggregates fuse into larger and larger spheres that form a separate phase by precipitation. The process lasts a few minutes or seconds. In the table the label “emulsion” refers to the cases where turbidity (milk-like) is retained for a longer period of time. A certain level of phase separation (visible lower phase) can be reached even in these cases, by centrifugation.] Coacervation takes place in a predetermined temperature range, and a solid phase different from the precipitate will form at a temperature typically lower than indicated in the range. In certain applications it is advisable to exploit the wide range of variations in the degree of hydration along with the different levels of interaction between particles.

### Example 3

### The Effect of the Concentration of the Surfactant on the Coacervation

Dilution series were prepared from SDS solutions in 1.2-ml reagent tubes. The vials contained decreasing amounts of SDS (50 μmol-0.5 μmol) and identical amounts (50 μmol-0.5 μmol) of agmatin sulphate complexing agent having double the charge of the SDS. Each samples has a total volume of 300 μl. After the addition of the complexing agent, apart from the vials containing a solution with the lowest concentrations, each solution became immediately turbid. After vortexing the tubes, the solutions were incubated for 10 min at ambient temperature, then centrifuged (3 min, 10000 g). The agmatin content of the equilibrium solution was determined by UV measurement (192 nm) of the samples taken from the upper phase, using a calibration series. (SDS has no extinction in the UV range.) The data in Table 2 show that up to the SDS concentration of 16.7 mM the concentration of the complexing agent is equal to the calculated value, i.e. the excessively added complexing agent is contained in the equilibrium solution, thus, the SDS is present in the coacervate. (This observation was verified by the result of measuring the SDS content of the equilibrium solution by the use of methylene blue.) At a value below 8.3 mM the relative value of the complexing agent content of the equilibrium solution rises abruptly. (This value is in good correlation with the 8.2-mM CMC value of the SDS.) Thus, at an SDS concentration of 8.3 mM only an insignificant amount of SDS is present in the coacervate. In the last two samples (3.3 mM and 1.7 mM) no observable coacervate can be found. The measurement justified the assumption that the polymolecular complex coacervation is a phenomenon that can be associated with surfactant micelles. Additionally, it provided information about coacervation taking place above CMC, and that the method is applicable well.

TABLE 2

| | Agmatin [mM] | | | | | | |
|---|---|---|---|---|---|---|---|
| | 166.7 | 83.3 | 33.3 | 16.7 | 8.3 | 3.3 | 1.7 |
| Equilibrial/ | 25.6% | 26.3% | 28.2% | 28.6% | 50.9% | 93.3% | 102.2% |
| original [%] | | | | | | | |

### Example 4

### The Effect of Salt Concentration on the Coacervation of Micelles

The relation between the coacervation and the changes in ionic strength was studied in solutions containing increasing amounts of NaCl. To NaCl solutions (260 μl), in 1.2-ml reaction flasks, SDS (20 μl, 0.5 M) then agmatin sulphate (20 μl, 1 M) were added. The process of the study was similar to the one described in Example 3. From the result it can be indirectly concluded that with a concentration up to 100 mM, the NaCl has no effect on the coacervation; nearly the total amount of the surfactant—complexing agent is in the coacervate. Above this concentration, the effectiveness of the phase separation deteriorates, since—similarly to the conventional complex coacervation—the ion-ion interaction between the salt and the micelle gets strengthened. The results are shown in Table 3.

TABLE 3

| | NaCl [mM] | | | | | |
|---|---|---|---|---|---|---|
| | 0 | 30 | 40 | 100 | 300 | 867 |
| Agmatin | 31.56 | 31.49 | 31.21 | 31.47 | 45.28 | 85.07 |
| (equilibrium | | | | | | |
| phase) [mM] | | | | | | |

### Example 5

### The Effect of pH Value on the Coacervation of the Micelles

The dependence on the value of the pH was studied under conditions identical with the ones described in Example 4, with the exception that the inventors used solutions with different pH values instead of applying solutions of different salt concentrations. From the results shown in Table 4 it can be concluded that polymolecular complex coacervation proceeds undisturbedly in the pH range between the values of the surfactant pKₐ and the pKₐ of the complexing agent. Besides theoretical consideration, i.e. both the surfactant and the complexing agent should be in ionized state, the assumption that pH dependence is determined by the pKₐ values is supported by the observation that coacervation did not take place either with butylamine hydrochloride at pH 8.8 or with aniline hydrochloride at pH 7.0.

TABLE 4

| | pH | | | | | |
|---|---|---|---|---|---|---|
| | 1.3 | 5.3 | 7.0 | 8.8 | 11.7 | 12.7 |
| Agmatin [mM] | 30.8 | 31.1 | 30.8 | 31.4 | 30.7 | 71.2 |

### Example 6

### Equilibrium of the Polymolecular Coacervation of the Surfactant Micelles

SDS solution (100 μl 0.5 M), then water (150 μl) were added into a 1.2-ml reaction flask. The addition of agmatin sulphate (50 μl, 1 M) resulted in coacervation.

To reach the equilibrium between the coacervate and the equilibrium phase the system was warmed up, vortexed, cooled down, then the phases were separated effectively by centrifugation. This procedure was repeated twice. Finally, the agmatin concentration of the equilibrium phase was determined by UV spectrometry. From the measured and the initial concentration values the approximate value of the stability coefficient was calculated on the basis of the general thermodynamic laws of the phase equilibrium. (Due to the inaccurate feature of volumetry only a rough estimation can be made.)
−lg Kₛ=2*lg[SDS]+lg[Agmatin] (1)

The approximate value of the stability coefficient is 1.13*10⁶. Under experimental circumstances described in the example, the values in Table 5 can be calculated by using the stability coefficient. However, the calculated values can only be applied for demonstrating the changes in the equilibrium, since the value of the coefficient was only approximately determined. The example also demonstrates that polymolecular complex coacervation—since it is a colloid system based on the self-assembly of small molecules—can be studied and optimized by using classical chemical methods.

TABLE 5

| Agmatin [mM] | SDS [mM] | SDS |
|---|---|---|
| initial conc. | equilibrium phase | equilibrium/initial |
| 3.3 | 16.3 | 9.8% |
| 33.3 | 5.2 | 3.1% |
| 166.7 | 2.3 | 1.4% |
| 333.3 | 1.6 | 1.0% |
| 500.0 | 1.3 | 0.8% |
| 1000.0 | 0.9 | 0.6% |

### Example 7

### The Effect of Protein-Surfactant Ratio on Polymolecular Complex Coacervation

SDS denaturates proteins and forms association complexes with them (through binding to micelles). Relative to micelles, the protein-SDS associate represents a new entity, since proteins contain specific ionic and hydrophilic groups that may affect the coacervation. Therefore, the parameter-dependence of the protein-surfactant associate can differ from that of the pure surfactant systems. Associates formed by surfactants with lypophilic compounds have a lypophilicity that is not higher than that of the original micelles, thus the parameters of the coacervation remain unchanged.

An SDS solution (100 μl, 10%) was measured to five 1.2-ml reagent tubes, then an increasing volume (10, 25, 50, 75 and 100 μl) of bovine serum albumin solution (BSA, 100 mg/ml) was added to each tube. The solutions were filled up to 200 μl, then agmatin-sulphate solution (40 μl, 1 M) was added to each of the tubes. When the complexing agent was added to the solution, it became turbid. After being vortexed, the tubes were incubated (10 min, ambient temperature), then centrifuged (2 min, 10000 g). The protein content of the equilibrium phase was measured by the Bradford method (at 595 nm). The results are shown in Table 6. From the results it is apparent that during coacervation the amount of protein that gets into the coacervate depends on the saturation rate of the surfactant. In the case of an unsaturated state, the aqueous solubility of the partly complexed protein inhibits the process of getting into the coacervate. When a sufficient amount of surfactant is present, the protein-associate can be brought to the coacervate with good efficiency, the rate of distribution allows efficient separation.

TABLE 6

| | BSA in | BSA in | | |
|---|---|---|---|---|
| | original | equilibrium | BSA | BSA |
| SDS/BSA | solution | solution | remaining/ | concentration |
| ration | [μg] | [μg] | total | [mg/ml] |
| 10 | 1 000 | 0.0 | 0.0% | 0.000 |
| 4 | 2 500 | 11.9 | 0.5% | 0.050 |
| 2 | 5 000 | 129.0 | 2.6% | 0.538 |
| 1.3 | 7 500 | 406.4 | 5.4% | 1.693 |
| 1 | 10 000 | 575.8 | 5.8% | 2.399 |

### Example 8

### The Effect of Salt Concentration and Certain Other Compounds on the Coacervation of Protein-Surfactant Associates

Systems containing BSA solution (10 μl, 100 mg/ml) and having NaCl in different concentration (or other compounds), SDS solution (20 μl, 0.5 M) and agmatin sulphate solution (20 μl, 1 M) (in a total volume of 300 μl) were coacervated. As the data in Table 7 show, the effect of NaCl is obvious above the concentration of 100 mM in the purely surfactant systems (Example 4), however, in the protein-surfactant associates the concentration is higher than 400 mM. A few compounds and buffers used in biochemistry were randomly selected and studied. The results are near to the detection limit which show that these compounds exert no significant effect; however, it appears that 2-mercaptoethanol, Brillant Blue R-250 and citrate buffer reduced, and other compounds slightly increased the concentration of the BSA in the equilibrium phase.

TABLE 7

| NaCl | Absorbance | | Concen- | Absorbance |
|---|---|---|---|---|
| [mM] | (A₂₈₀-A₃₂₀) | Other compound | tration | (A₂₈₀-A₃₂₀) |
| 0.0 | 0.0020 | Mercapto-ethanol | 0.3% | 0.0001 |
| 33.3 | 0.0009 | Brillant Blue R-250 | 0.01% | 0.0009 |
| 83.3 | 0.0014 | EDTA pH 8.0 | 50 mM | 0.0036 |
| 166.7 | 0.0015 | Glycerol | 10% | 0.0060 |
| 333.3 | 0.0007 | Arginine pH 8.0 | 40 mM | 0.0039 |
| 666.7 | 0.0030 | Na-citrate pH 5.0 | 100 mM | 0.0012 |
| 1333.3 | 0.0173 | | | |

### Example 9

### The Effect of pH and the Surfactant/Protein Ratio on the Coacervation of Different Protein-Surfactant Associates and on the Separation of the Proteins

The inventors studied the polymolecular complex coacervation of two proteins with different characteristic features. While BSA (Ms.: 66 500, pI 4.8) is an acidic, carbohydrate-free protein, avidine (Ms.: 68000, pI 10.5) is a definitely basic protein that contains 10% carbohydrate and has four subunits. A system (with a total volume of 300 μl) containing SDS (20 μl, 0.5 M), agmatin sulphate (20 μl, 1 M) and protein (0.1-10 mg) was coacervated without adding buffer or with a buffer (15 μl, 1 M Na-acetate buffer (pH 5.3), or 15 μl, 1 M Tris buffer (pH 8.8)). The coacervation was performed as described above, and the protein content of the equilibrium solution was determined by measuring the absorbance at 280 nm (corrected with the value measured at 320 nm). The results are shown in Table 8. Proteins saturated with the surfactant can be brought to coacervate (see Example 7). The proteins covered by the surfactant can be distinguished only by their molecular weight. The results show that proteins that are not saturated with the surfactant get into the coacervate in a higher ratio when the pH is below or near their isoelectric point compared with the case when the pH is higher than their isoelectric point. If the surfactants are used in a concentration lower than that of the saturated solution, the specific characteristics of the proteins become apparent. This can be utilized at separation of the proteins from each other. The data presented in Table 8 show that at neutral pH and when 5 mg of protein is used, the equilibrium solution contains 2.147 mg of BSA but only 0.149 mg of avidine. This is a significant difference especially in chromatographic applications. To determine whether the proteins will separate in the coacervation of the system containing the two proteins together—i.e. the individual protein molecules will be packed separately into the surfactant shell—a solution containing 2.5 mg BSA and 2.5 mg avidine together (pH 7.0) was coacervated. By checking the UV spectra it was determined that a characteristic shoulder can be seen at 291 nm in the avidine spectrum. Compared to each other, BSA gives a higher value at 278.5 nm while at 291 nm avidin results in higher absorbance. The ratio of absorbance values measured at 278.5/291 nm is 1.923 in the case of pure BSA, 0.804 in the pure avidine and it is 1.804 in the BSA/avidine system, respectively. From these data it is easy to calculate that BSA constitutes 89.3% of the protein content of the equilibrium solution. This value would be 93.5% if we calculated it on the basis of a pure system, i.e. the BSA/avidine mixed system reproduces the value calculated from pure systems with an accuracy of 95.5%. Bearing the inaccuracy of the measurement in mind, the inventors concluded that in the coacervation of the protein mixtures the phase-distribution of the individual proteins is identical with the values obtained in the coacervation of the individual proteins. In other words, in systems that contain more than one proteins, the individual protein molecules will be packed separately into the surfactant micelles, i.e. the elemental units of the coacervate are formed by an individual protein molecule and the surfactant micelles associated therewith.

TABLE 8

| Original | | | | | | |
|---|---|---|---|---|---|---|
| value | Equilibrium solution | | | | | |
| protein | BSA [mg] | | | Avidine [mg] | | |
| [mg] | pH 5.3 | pH 7.0 | pH 8.8 | pH 5.3 | pH 7.0 | pH 8.8 |
| 0.1 | −0.018 | 0.001 | 0.007 | 0.009 | 0.003 | 0.067 |
| 1 | −0.004 | 0.028 | 0.071 | −0.002 | 0.010 | 0.066 |
| 2 | −0.006 | 0.064 | 0.476 | 0.004 | 0.010 | 0.134 |
| 3 | 0.014 | 0.295 | 1.206 | 0.010 | 0.014 | 0.372 |
| 4 | 0.030 | 1.034 | 2.128 | −0.001 | 0.060 | 0.654 |
| 5 | 0.076 | 2.147 | 3.197 | 0.018 | 0.149 | 1.167 |
| 10 | 7.563 | 9.417 | 7.804 | 2.748 | 3.973 | 4.563 |

### Example 10

### The Repeatability of Polymolecular Complex Coacervation, Concentrating Proteins and the Recovery of the Proteins from the Coacervate

SDS solution (20 μl, 0.5 M), water (230 μl) and finally agmatin sulphate (10 μl) were added to the BSA solution (10 μl, 100 mg/ml) resulting in the immediate start of the coacervation. After 5 min incubation, the clean separation of the coacervate from the equilibrium solution was carried out by centrifugation (3 min, 10000 g). The equilibrium solution was pipetted to another flask and SDS solution (20 μl, 0.5 M) was added to it, then a second coacervation event was initiated with agmatin sulphate (15 μl). SDS (20 μl, 0.5 M) was added to the original coacervate with a volume of 301 (i.e. the original solution concentrated to one-tenth in volume)—which was separated from the equilibrium solution—and the mixture was incubated at 37° C. for 10 min (with shaking). At the end of the incubation the coacervate and the SDS formed a homogenous phase. Then water (200 μl) was added to the mixture and the protein content of the clean solution was measured spectrophotometrically. According to the result 94.2% of the original amount of protein was recovered from the coacervate. The measurement verified that the majority of the proteins were present in the coacervate, i.e. during the process of coacervation the protein concentrated to the one-tenth of the original volume.

### Example 11

### Removal of Proteins by Polymolecular Complex Coacervation

In many cases, the actual task is to remove and not to recover the protein from a solution. Several such cases can be encountered, such as, after enzymatic reactions, the removal of the unnecessary enzyme. In the electrophoretic analysis of nucleic acids the proteins are interfering factors and their removal is a very frequent task. The removal of degradative enzymes (proteins) from a solution can assure the biological stability of the remaining materials. From among the enzymes causing degradation it is the group of ribonucleases (RNAse) that is worthy of note. These enzymes show enormous resistance to both chemical and thermal effects. In an experiment the present inventors studied the removal of ribonuclease A enzyme by polymolecular complex coacervation. Applying a biological model, a solution called solution A (150 μl) was prepared from the following components: BSA (1 mg), RNAse A (25 μg), 1 M Tris buffer, pH 8.0 (30 μl) and 0.5 M EDTA, pH 8.0 (10 μl), then solution B was prepared by adding SDS (150 μl, 10%) and mercaptoethanol (10 μl) to solution A. Coacervation was effected in solution B by the addition of agmatin sulphate (60 μl, 1 M). The equilibrium solution C was separated and re-coacervated by adding SDS (100 μl 10%) and agmatin sulphate (40 μl, 1 M). The resulting equilibrium solution D was obtained from the second coacervate. RNS transcript labelled with [α-P32] CTP (2 μl) was added to each sample (10 μl) collected from the solutions. According to the samples analyzed by TLC while the RNS in solution A was degradated in 5 min, the degradation in solutions B, C or D accounted for only a small percentage (it is likely that the degradation is due to the radiolysis of the sample). The results show, that a suitable amount of SDS supplemented with mercaptoethanol effectively inhibits the ribonuclease A enzyme, and the polymolecular complex coacervation effectively makes the solution protein-free already in one single step.

### Example 12

### Separation of Protein and DNS by Polymolecular Complex Coacervation

Proteins and nucleic acids are macromolecules of colloid size which are considered to be the most important substances of living organisms. Their separation from each other by using a colloid-based method is a task of great importance. To demonstrate the efficacy of polymolecular complex coacervation a study was performed with a DNA of an especially high molecular weight (10-15 million Da). A 270-μl mixture of BSA (1 mg, DNA (400 μg), NaCl (30 μl, 1 M), Tris buffer, pH 8.0 (30 μl, 100 mM), EDTA, pH 8.0 (6 μl, 0.5 M) and SDS (100 μl, 0.5 M) was coacervated by the addition of agmatin sulphate (40 μl, 1 M). To protect the DNA, homogenization was performed without vortexing and by only rotating the reaction flask. After 5 min of incubation, separation of the phases was enhanced by centrifugation (3 min, 10000 g). The absorbance of samples taken from the equilibrium solution was measured at 260, 280 and 320 nm. On the basis of the measured values corrected with the values taken at 320 nm, the equilibrium phase was free of proteins (the A260/A280 ratio was 1.85) and 88.7% of the originally added DNA was recovered. The measurement justified that the complexed surfactant-protein colloid brought to the coacervate can be separated very well from the DNA—even from the extremely huge, colloid-size DNA—remaining in the equilibrium solution.

### Example 13

### Isolation of Nucleic Acids from Biological Samples by Polymolecular Complex Coacervation

The isolation of DNA by the use of SDS is one of the oldest and most wide-spread techniques. Since the technique suits well to the object of the invention, the inventors tried to take advantage of applying it. By using the conventional method, 3 g of onion was homogenized in a mixture of SDS (2 ml, 0.5 M), NaCl (0.45 ml, 2 M) and water (3.55 ml) and the homogenate was incubated at 60° C. for 5 min. After centrifugation, the sample taken from the supernatant was subjected to coacervation by the addition of agmatin-chloride. However, coacervation did not occur even in the excess of the complexing agent. The inventors realized that the reason for this was represented by the problems that, on one hand, the majority of the SDS added to the sample was in a bound state in the centrifugation sediment, and, on the other hand, the supernatant contained proteins that were not saturated with SDS. The experiment was repeated with double the amount of SDS and by adding agmatin-chloride (100 μl, 1 M) to the sample (600 μl) taken from the supernatant. In this case the system clearly coacervated. The sample drawn from the equilibrium phase was free of proteins (determined by the Bradford method). Measured by UV spectrometry, the value of the corrected A260/280 ratio was 2.75, which showed that besides the nucleic acids other UV absorbing materials are also present in the sample. Polymolecular complex coacervation effectively removes the proteins and lipids that represent the two most problematic groups of compounds in the application of nucleic acids. Additionally, polymolecular complex coacervation effectively removes several other materials that can associate with the surfactant micelles, however, it does not mean that only nucleic acids will remain in the equilibrium solution. The other compounds (possibly with small molecular weight) remaining in the equilibrium solution do not disturb the direct use of the equilibrium solution because the problem is caused by the proteins, that can bind and degrade the nucleic acids and lipids forming a separate phase. Due to the equilibrium correlations of the coacervation the SDS remains in the solution only in traces. According to our measurements agmatin, which is an amino acid derivative, and which occurs also in nature at a concentration below 50 mM, does not interfere with the enzymatic reactions. Of course, there is a possibility for the equilibrium solution to be purified. By using gel filtration or precipitation with alcohol pure nucleic acid can be obtained. Alcohol (in a 2.5-fold volume, −20° C.) was added to the equilibrium solution. After incubation (at −20° C.), the precipitated nucleic acid was spinned down and dissolved in TE solution (10 mM). The value of the 260/280 ratio was 1.89 showing the presence of pure nucleic acid solution.

### Example 14

### Determination of the Parameters of Polymolecular Complex Coacervation in the Case of Isolation and Purification of Nucleic Acids from Biological Samples

In the process of isolating nucleic acids it is a general task to reach the most efficient removal of the proteins. As has been shown in Example 8, the surfactant/protein ratio has a great impact on the distribution of the proteins between the equilibrium solution and the coacervate. In several cases the protein content of a given solution is well known (e.g. in enzymatic reactions), so it is the concentration of the surfactant, necessary for the process, that should be determined. The situation is more complex when nucleic acids should be isolated from different cells or tissues. Since the added surfactant binds not only to the proteins but also to several other compounds, therefore, the mere knowledge of the amount of protein in the system is not sufficient. If the necessary amount of the surfactant has already been determined for a given cell or tissue, in the subsequent experiment the necessary amount of the surfactant can be calculated on the basis of the amount of the given cell or tissue. In the case of a sample with unknown composition the necessary amount of the surfactant can be determined by the simple technique of using coacervation itself. By the addition of a protein stain to the system the colour of the two phases indicates the distribution of the proteins, i.e. the appearance of a colour in the equilibrium solution indicates that the amount of the added surfactant was smaller than would have been necessary for “packaging” all of the proteins. The necessary amount of the surfactant can also be determined on the basis of the observation of the coacervation. By increasing the amount of the complexing agent relative to the amount of the surfactant, the viscosity of the solution becomes higher. When a further amount of complexing agent is added to the system, the coacervate appears and its volume will decrease by the addition of even more complexing agents. Finally, it reaches a level characteristic of the given complexing agent and the level will not decrease even by addition of a still further amount of complexing agent. The process can be explained by the decrease of the hydration.

Calf's liver was powdered in liquid air in a porcelain mortar. Powdered liver (622.7 mg) was added to SDS solution (2 ml, 0.5 M) (in small portions to the surface of the solution). The lysate (200 μl) measured into four reaction tubes was mixed with Brillant Blue R-250 solution (50 μl 0.1%) and with SDS solution (0, 100, 200, 300 μl, 0.5 M), the mixture was coacervated with agmatin-chloride (200 μl, 1 M) at 37° C. for 5 min, then the solutions were centrifuged (15000 rpm, ambient temperature). The first equilibrium solution was medium blue, the second was pale blue, and the last two were colourless. The equilibrium solution was clean in each case, non-lysed cells and cellular organs were brought into the coacervate. However, the coacervate formed with lipids was found on the top of the equilibrium solution and it could be easily eliminated by the addition of some chloroform which does not interfere with coacervation. The nucleic acids were precipitated from the last two equilibrium solutions by using −20° C. ethanol. The spectrophotometric determination revealed that the nucleic acids were recovered by coacervation with good efficiency (15.1 and 15.4 μg/mg, respectively) and purity (A260/280 is 1.85 and 1.89, respectively). Similar results were obtained in the cases when the calf's liver was homogenized with the same amount of solid SDS, and the frozen liver/SDS powder was mixed into TE buffer, then the mixture got coacervated.

The example above is intended to illustrate the applicability and limits of the coacervation. To avoid the possible side effects only a “minimal” system containing the necessary materials was used without any optimization. As Example 7 shows, polymolecular complex coacervation also works almost unaltered in the presence of several other compounds. Thus, there is no drawback of carrying out the procedure by the addition of the compounds generally used in the isolation of nucleic acids (such as buffers, chelating agents, reducing agents, compound controlling pH and ionic strength). The example also shows that a good result can be achieved only by using the compounds absolutely necessary for the coacervation. Further additives and the optimization of the process can enhance the yield of the nucleic acid isolation and the integrity of the nucleic acids without changing the essence of the procedure based on the coacervation.

### Example 15

### Production of Protein Containing Microcapsules, Microemulsions and Dispersions as well as Films by Polymolecular Complex Coacervation

In the protein type of interaction both the lipophylic and hydrophilic portions of the micelles have specific roles. Depending on their sequence the polypeptide chains may be embedded, may surround the micelles or the micelles may cover them. In essence, the procedure of forming microcapsules does not differ from the processes of the phase separation applications. Primarily, the selection of a complexing agent is the step whereby the different applications should be also taken in account. Thus, in the production of microcapsules it is preferred to use complexing agents which provide solid shell and micelle-crystallize at the temperature of application. From among the complexing agents it is preferred to use the ones that provide a relatively large amount of coacervate, i.e. those whose elements have a relatively big hydrate shell, thus ensuring that the individual particles can be dispersed. However, for the production of micro- and nano emulsions, complexing agents that do not micelle-crystallize at the application temperature should be used. The process follows a general scheme: a complexing agent should be added in about equimolar quantity or in an amount higher than the equimolar to a self-assembling colloid system containing both an associate of surfactant in a concentration higher than the cmc value and compounds attached directly or indirectly to associate. The solution should be optionally warmed until coacervation takes place, or the process is carried out ab ovo at this temperature. After coacervation the coacervate will be cooled and dispersed (e.g. by stirring). The dispersibility, the size of the nanocapsules, and their attachment can be modified by the addition of supplements or by the selection of the appropriate complexing agent and taking the right amount of it. Since the microcapsules prepared by using the method of the invention are lypophilic, they can be kept in solution by the aid of hydrophilic colloids (such as polyvinyl alcohol, polyvinyl pyrrolidone, etc.)

In the case of several complexing agents the size of the particles obtained through cooling and dispersing the coacervate was in the micrometer range. Merely on the basis of their size they cannot be regarded as units composed of a polypeptide chain and the micelle attached to it, as has been found in the original solution. Due to the self-assembling nature of the system, the inventors, however, have obtained structures of identical size. It is interesting to note the phenomenon that while the globular BSA produced globular structures, the gelatine listed in the group of linear proteins and the synthetic polyethylene imine contained elongated, bunched parts (bound to each other like a mop of hair) displaying the shape of the original building units. In the case of certain complexing agents, the capsules were of invisible size when observed through a light microscope; it was only after the sample had dried that the nano-capsules got attached, thereby displaying visible formations.

Water (200 μl), then guanidinium chloride (100 μl, 1 M) were added to the 1:2 (v/v) mixture of BSA solution (100 μl, 100 mg/l) and SDS (0.5 M). The solution containing some white unstructured precipitate was incubated at 60° C. for 5 min. During the incubation the solution separated into two transparent liquid phases. The coacervated solution was homogenized by vortexing, then incubated again at 60° C. For the collection of the colloid particles floating in the equilibrium solution, the system was centrifuged (2 min, 10000 g). According to the Bradford assay performed with a sample taken from the equilibrium solution was free of proteins. The reaction vial kept at the temperature of 60° C. was placed in liquid nitrogen for a short time and frozen in it. After the sample had been thawed, the equilibrium solution was removed and the weight of the solid coacervate was measured (74 mg). Water (100 μl) was pipetted to the white, solid coacervate and the mixture was homogenized by vortexing. The sample, taken from the macroscopically milk-like, homogenous microdispersion, was observed under microscope. Homogenously distributed, micron-sized round structures could be observed. The brownish light, which could be observed in the microscopical examination of the white product, suggests the presence of invisible particles with a size of the magnitude of the wavelength of the light. The majority of the product could be sedimented by centrifugation and a part of the product could be filtered out. The inhomogenity of the particle size distribution shows that, besides the major portion that falls in the micrometer size range, nanometric particles could also be produced. The study was repeated with gelatine and polyethylene imine, too. The only discrepancy was represented by the shape of the capsules. The shape of the capsules in question, which are produced from natural or synthetic, linear polymers, displayed a characteristic elongated form. It was possible to follow the way of the proteins through the addition of Brillant Blue protein stain. During coacervation the lower phase was dark blue while the upper phase had a very pale blue colour. After the centrifugation of the system, the equilibrium solution became colourless. After the coacervate had been dispersed, the micro-dispersion got a typical silky pale blue colour.

The above basic study was repeated in a way that the system contained not only guanidinium chloride (100 μl, 1 M) but 4-guanidinobenzoic acid-4-nitrophenyl ester hydrochloride (100 μl, 10 mM), too, as complexing agents. The very pale butter yellow product changed its colour to sulphuric yellow when NaOH solution was dropped into it. The change in the colour indicated that the guanidine derivative—which otherwise is used as trypsine substrate—got embedded into the micelles.

Water (200 μl) and SDBS solution (100 μl, 0.5 M) was added to a BSA solution (20 μl, 100 mg/ml), then a defined amount of arginine hydrochloride solution (100 μl, 1 M) was added to it. Homogenization resulted in a milk-like emulsion. A sample (5 μl) taken from the emulsion was diluted with water (20 μl) and spread on a microscopy slide. After drying, a completely transparent, homogenous, lacquer-like coating film was obtained.

### Example 16

### Production of Microcapsules Containing Oily Substances by Polymolecular Complex Coacervation

In an other type of surfactant micelle interactions, oily substances interact with the lipophylic portion of the micelles. Lypophilic substances with homologous characteristics dissolve in the internal portion of the micelles and, thus, resulting in their quasi swelling. The inventors observed, that some parts of the coacervate that composed of oil-containing microcapsules tend to surround the excess microdrops that had not been incorporated by surfactant micelles and form a solid layer on their surfaces, thus, providing an opportunity for microcapsulating certain oily substances in an amount higher than the equilibrium value. It is essential to apply complexing agents micelle-crystallizing at the application temperature, otherwise, the dynamically changing system will release the oil incorporated in the amount exceeding the equilibrium level. By the use of the micelle-crystallizing complexing agent, the self-assembling system will freeze in a given state leading to the termination of the further transport of substances. The microcapsulation of oily substances differs from the method described in Example 15 in certain steps: the coacervate is emulgeated at the coacervation temperature, and the end-product is obtained by the quick cooling of the emulsion. If the efficiency of the cooling is not sufficient, there is a chance of triggering the formation of regular crystals, which leads to the disruption of the microcapsules and the release of their content.

Paraffin oil stained red with Sudan III stain (25 μl), SDBS (100 μl, 0.5 M) and arginine hydrochloride (300 μl) was coacervated at 75° C. The mixture was homogenized by vortexing while warm and the reaction flask containing the emulsion was immersed in liquid nitrogen until its content became frozen. After thawing and filtering, a creamy pink product was obtained. On the basis of the microscopic picture, the product contained inhomogenuosly distributed, coated microdrops (with a size of up to 80 μm). By more efficient homogenization and by using bigger shearing force more homogeneous and smaller capsules can be obtained, although even this product could sufficiently keep the oil. Similar results have been achieved in studies with virgin olive oil/SDS/guanidine hydrochloride and some other systems.

### Example 17

### Production of Complexed Reverse Micelles

It is well known that in an apolar solvent the surfactants form reverse micelles in which the surfactant molecules are in the position where their hydrophilic part oriented to the internal part of the micelles while the hydrophobic part turns towards the apolar solvent. Similarly to the system composed of normally oriented micelles and the oil “dissolved” in them, in the apolar solution the polar compounds migrate to the internal hydrophilic environment of the micelles. The micelles get stabilized, provided they contain a polar solution in an amount depending on the characteristics of the surfactant. The inventors found that such reverse micelles can be complexed similarly to the normally oriented micelles. Complexed micelles can be produced in two ways: either the aqueous solution of the complexing agent is added to the surfactant dissolved in apolar solvent, or the complexed surfactant is dispersed in the apolar solvent and the reverse micelles are formed by the addition of water. The use of surfactant/complexing agent systems that form micelle-crystals at ambient temperature is especially important, since reverse micelles having solid wall can be produced with them.

Guanidinium dodecylsulphate (regular crystals, 13.8 mg) was suspended in the mixture of petrolether (0.5 ml) and chloroform (0.5 ml). After the addition of NaCl (20 μl, 0.5 M), the mixture was warmed up to 60° C. The obtained water-clear, homogenous solution congealed into a whitish coloured gel when cooled to ambient temperature. The inventors think that the temperature-dependency of the interaction between the complexing agent and the surfactant molecules is identical in the reverse micelles with that of the normal micelles. By warming, the gel repeatedly transforms into a homogenous solution at 60° C., then it forms a whitish coloured gel again upon cooling. The gel retained its form for several days at ambient temperature, thus, it can be stably maintained. By the addition of chloroform the gel structure can be disrupted and the solid sample floating on the surface of the solution has a microscopical image that is similar to that of the regularly oriented micelles.

### Example 18

### Phase Separation of Nucleic Acids by Using SDS-Spermine Coacervate

Depending on the conditions the SDS-spermine system can be in a state of solution, coacervate or precipitate. For the phase separation of nucleic acids the most feasible strategy may be to mix the solution of SDS-spermine composition with the system containing nucleic acids and, simultaneously, to bring the medium into the coacervate (e.g. by increasing the dielectric constant of the medium), then to separate the nucleic acid containing coacervate from the equilibrium phase by the use of a physical method (such as sedimentation, centrifugation, filtration, spooling to a glass rod, etc.). The nucleic acids can be recovered from the isolated coacervate by terminating the multiparameter equilibrium state, e.g. by disrupting the bonds between the nucleic acid and the coacervate, or bringing the coacervate into solution, etc.

Mirorrly clear, light blue “coacervating solution” was prepared by mixing 30 μl 1% SDS, 50 μl 0.1% CBB, 25 μl propanol and 20 μl 100 mM spermine. The “coacervating solution” was added to a solution containing 50 μg DNA, 120 μl 10 mM Tris-HCl (pH 8.0), 1 mM EDTA (pH 8.0) and 100 mM NaCl. Subsequently to the addition, the coacervation immediately starts, which is indicated by the appearance of an opalescent solution containing light blue spheres. By careful agitation the coacervate will concentrate in the middle of the solution in the form of a clew. After a few seconds' process, a 200-μl colourless solution was obtained in the middle of which a cca. pea-sized blue clew-like structure was floating (FIG. 10). The equilibrium solution surrounding the blue clew-like coacervate was pipetted out, and the clew-like coacervate was washed twice with 200 μl distilled water. Subsequently to the washing with distilled water, the clew-like coacervate comprising the DNA can be stored in an unchanged form. The coacervate can also be separated by centrifugation which leads to a viscous block on the bottom of the reaction vial, and this form is more suitable for storing.

The DNA was recovered by adding 25 μl distilled water, 10 μl 2M NaCl, 10 μl 1% SDS solution and 5 μl i-propanol to the coacervate. The dissolution was enhanced by warming and agitating. Following dissolution, 50 μl 400 mM NaCl solution was added to the homogenous blue solution and the reaction vial was kept on ice for a few minutes. The resulted precipitate was removed by filtration or centrifugation. A five-times volume of 80% ethanol (equal volume of distilled water and four-times volume of ethanol) was added to the supernatant. The precipitated DNA was resolved in 10 mM Tris-HCl, 1 mM EDTA (pH 8.0).

### Example 19

### Interaction of Nucleic Acids with Coacervates Formed by the Interaction of Multivalent Complexing Agents and SDS

For studying the interaction of nucleic acids with coacervates formed by the interaction of multivalent complexing agents, the following experiment was performed. A stock solution was prepared and divided into five parts. Each reaction vial contained a solution with the same composition: 20 μl 2.5 mg/ml DNA, 10 μl 0.5 M SDS, 25 μl 2 M NaCl, 25 μl 0.1% CBB and 370 μl distilled water. Different complexing agents in equal volume were added to each reaction vial. The complexing agents were the following: neutral solutions of 200 mM ethylene-diamine, 200 mM agmatin, 100 mM spermidine, 100 mM spermine and 100 mM tetraethylene-pentamine. After the addition of complexing agents, the mixtures were homogenized, incubated for 5 min at 60° C., then centrifuged (10 min, 4° C., 10000 g). In each case the blue coacervate was found on the bottom of the reaction vial, covered with colorless equilibrium solution (except the ethylene diamine). (Probably, due to the weak complexing feature of the ethylene diamine, in this case the equilibrium phase was light blue. The UV spectra of the original solution and the samples collected from each equilibrium solution were recorded and evaluated. (Table 9). The results show that the coacervate formed of the bivalent ethylene diamine and the agmatine complexing agents with SDS does not interact with the DNA. The trivalent spermidine with high yield, the tetravalent spermine and the pentavalent tetraethylene pentamine quantitatively contain the DNA in the coacervate.

TABLE 9

| | Complexing agents | | | | |
|---|---|---|---|---|---|
| | | Ethylene | | | Tetraethylene |
| | Agmatine | diamine | Spermidine | Spermine | pentamine |
| Number of complexing | 2 | 2 | 3 | 4 | 5 |
| groups | | | | | |
| A₂₆₀ of the | 100.8% | 102.0% | 7.5% | 0% | 0% |
| equilibrium solution/ | | | | | |
| A₂₆₀ of the | | | | | |
| original solution | | | | | |

### Example 20

### Isolation of Plasmid DNA by Colloid Phase Separation from E. Coli Bacteria

The phase separation of micellar colloid solutions according to the invention is an effective tool for recovering nucleic acids from different biological samples. Coacervation performed with monovalent or bivalent complexing agent (such as agmatin) and anionic detergent (such as SDS) can bring the proteins and lipids into the coacervate and the nucleic acids into the equilibrium solution. Optionally, the use of specific digesting enzymes (RNase, DNase) applied for isolating the two large groups of nucleic acids, such as ribo- and dezoxyribonucleic acids, fit particularly well in the colloid phase separation method, since following the breaking of nucleic acid types unnecessary for a given purpose, together with other proteins the digestion enzymes will also get into the coacervate and will not contaminate the final product. Nucleic acids obtained in such a way are free of lipids and proteins and have a quality that allows them to be used for several purposes (such as screening). The equilibrium solution of the coacervate contains not only nucleic acids but usually also other compounds with small molecular weight (e.g. amino acids, nucleotides, metabolites, complexing agent, etc.) from which the nucleic acids can be separated (e.g. by precipitation with alcohol) efficiently.

A second coacervation performed in the equilibrium solution of the first coacervation provides particularly high purity nucleic acid. Provided the second coacervation is performed with a trivalent or multivalent complexing agent (e.g. spermine) and an anionic detergent (e.g. SDS), the nucleic acids get into the coacervate while all the contaminants will be in the equilibrium solution. The spermine can condense only nucleic acids larger than a specific size (approximately 100 bp), thus nucleic acids can be separated also from oligonucleotides (such as PCR primers, end products of digestion with RNAse and DNAse, labelled nucleotides, etc.).

The most widely-spread method for recovering plasmid DNA is the alkaline denaturation. In this method lysing the cells with alkaline SDS leads to the simultaneous denaturation of nucleic acids. The two types of nucleic acids react differently to the change in pH due to the effect of highly concentrated salt solution with low pH (3 M acetate) that is added to the lysed sample. The closed circular plasmid DNA can be completely renaturated and it gets into solution, however, the chromosomal DNA and the bacterial DNA comprising jumbled strains together with the precipitated proteins and cell debris form a precipitate.

The disadvantage of the method is that the proteins are only partially removed by salt precipitation, therefore the plasmid DNA is contaminated with proteins. Another disadvantage of the method is that the plasmid DNA solution also contains a significant amount of lipopolysaccharide (LPS); thus, for example, it provides a product inadequate for transfection. By performing the method according to the coacervation of the invention, a product completely free of proteins and pyrogens can be obtained.

The isolation of plasmids is performed in a kit like manner, i.e. by using prepared solution compositions:

| | |
|---|---|
| F1: | 50 mM Tris-HCl, pH 8.0 |
| | 10 mM EDTA, pH 8.0 |
| | 0.1 mg/ml RNase A |
| | 50 ppm CBB dye |
| F2: | 200 mM NaOH |
| | 1% SDS (w/v) |
| F3: | 70 mM Agmatin-sulphate |
| | 350 mM acetic acid |
| T1: | 40 mM SDS |
| | 40 mM Spermine |
| | 0.01% CBB (w/v) |
| | 30% isopropanol (v/v) |
| T2: | 200 mM Tris-HCl, pH 8.8 |
| | 200 mM NaCl |
| | 20% isopropanol (v/v) |
| T3: | 400 mM NaCl |

The plasmid extraction study was performed on DH5α E. coli line containing pBluescript KS vector. One milliliter sample of bacteria cultured overnight in LB medium containing 100 μg/ml ampicillin was centrifuged (6000 g, 1 min, 4° C.) in a 1.2 ml reaction vial. After the removal of the supernatant, the pellet was resuspended in 50 μl (F1) solution. The homogenous distribution of the blue stain indicated whether the suspendation was sufficient. 150 μl of (F2) solution was added to the homogenous suspension, then it was carefully homogenized without vortexing. After 5 min incubation 150 μl (F3) solution was added to the reaction vial and its content was homogenized by rolling the vial a few times. The coacervate containing genomial DNA and cell debris formed a ball-like, blue aggregate in the middle of the solution. The reaction vial was chilled for 5 min on ice, then centrifuged (15000 g, 1 min, 4° C.). The water clear solution (242.4 mg) well separated from the light blue solid precipitate and was measured and transferred into a fresh reaction vial. 85 μl of (T1) solution was added to the solution and the mixture was carefully homogenized. The fibrous, blue coacervate containing the plasmid DNA was collected by centrifugation (15000 g, 1 min, 4° C.). After decanting the supernatant, the coacervate in the reaction vial was washed with 400 μl distilled water. Following the complete removal of the washing water, 25 μl (T2) solution was added to the reaction vial. By dissolving the coacervate a homogenous blue solution was obtained. The dissolution was accelerated by vortexing and warming. Coacervation was initiated by the addition of 50 μl (T3) solution (the solution became turbid). The reaction vial was chilled for 5 min on ice, then centrifuged (15000 g, 1 min, 4° C.). The water-clear solution covering the dark blue coacervate was transferred to another reaction vial by pipetting, then the solution was diluted twofold with distilled water. The plasmid DNA was precipitated with alcohol, centrifuged, dissolved in 50 μl TE (pH 7.5) buffer and labelled as (P).

The protein content of the solution was determined by the Bradford method, while the pyrogen content was checked with LAL test. Both determinations gave negative results. The quantitative and qualitative evaluation of the plasmid DNA obtained by the method of the invention was checked by agarose electrophoresis (control: Hind III digested λ DNA marker (M)) and UV spectrophotometry. The yield was 13.0 μg and the A260/280 ratio was 1.81. The results confirmed that the method provides a high-purity product with a high yield, and the product is suitable for infection.

### Example 21

### Recovery of Nucleic Acids from Agarose Gel by Colloid Phase Separation, Storage of Nucleic Acids in a Coacervate

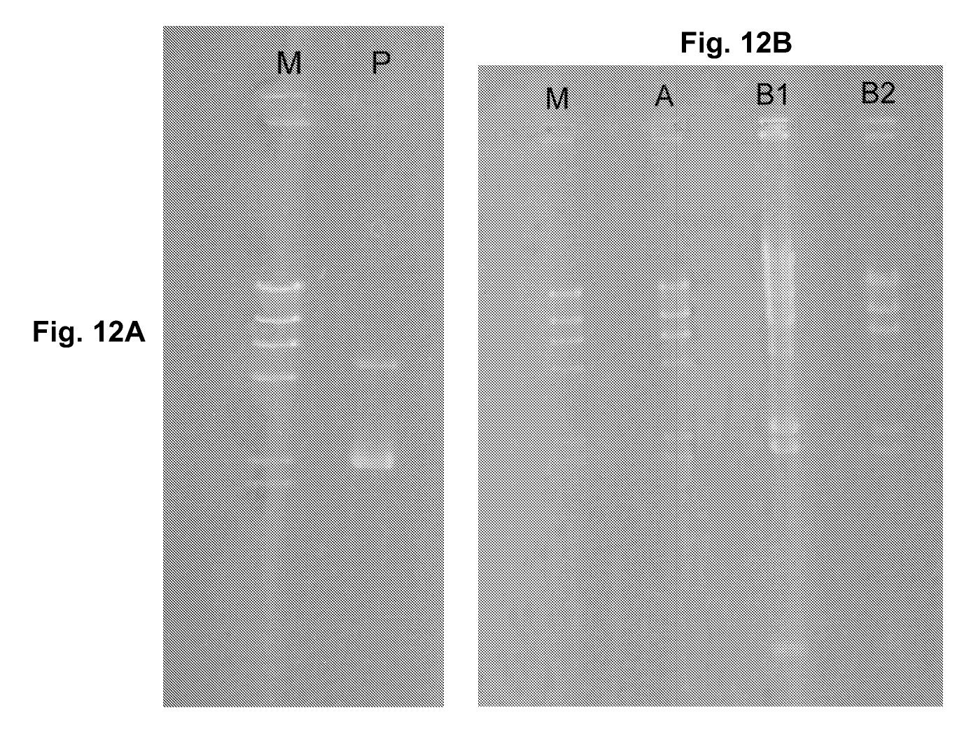

The recovery of nucleic acid samples from a gel following agarose gel electrophoresis is a frequent task. The colloid phase separation of the invention is also suitable for this purpose. The experiments were performed with Hind III digested λ DNA molecular weight marker (245 μg/ml, Sigma). In two parallel experiments 2 μl (Sample A) and 20 μl (Sample B) marker solution, i.e. 0.49 μg, and 4.9 μg DNA, were measured and added to two reaction vials. The marker solution was adjusted to 100 μl by the addition of 1% low melting point agarose gel melted at 70° C. (The 1% agarose gel was prepared in 1×TBE buffer.) Following homogenization. the gel containing the DNA was left to solidify then remelted at 70° C. By using the solution of Example 20, 25 μl (T1) solution was added to both reaction vials and the contents of the vials were slightly homogenized. The blue colored coacervate obtained was collected on the bottom of the vials by centrifugation (15000 g, 5 min, 4° C.). The agarose gel covering the coacervate was left to solidify and the samples were stored for a week at ambient temperature (FIG. 11). Following the one-week storage, the gel was melted at 70° C. and removed. The residual coacervates were washed twice with distilled water which was warmed up to 70° C. after the addition. Following the complete removal of the washing water, 25 μl (T2) solution was added to the reaction vials. By dissolving the coacervates, homogenous blue solutions were obtained. The dissolution was accelerated by vortexing and warming. Coacervation was initiated by the addition of 50 μl (T3) solution (the solutions became turbid). The reaction vials were chilled for 5 min on ice, then centrifuged (15000 g, 1 min, 4° C.). From the 75 μl of water clear solutions covering the dark blue, nucleic acid free coacervate, 50 μl were pipetted to the new reaction vials, and 5 μl of sample (B) was kept for electrophoresis (labelled with B2). 50 μl of distilled water and 200 μl of ethanol were added to the solutions pipetted into the new reaction vials. Samples were kept at −20° C. for one hour, then centrifuged (15000 g, 1 min, 4° C.). The alcohol-precipitated DNA was dissolved in 5 μl TE (pH 7.5) buffer and labeled with (A) or (B1). Samples (A), (B1) and (B2) were checked by agarose gelelectrophoresis (FIG. 12.B). 1 μl of the original solution served as control, thus the DNA content of sample (A) theoretically corresponds to 1.5-times of that of the control. The DNA content of the (B1) and (B2) is 15-times and 1.33 times of that of the control. By observing the ethidium bromide stained gel we assessed that the pictures of the (A) and (B2) are completely equivalent and their intensity is slightly beyond the intensity of the control. The intensity distribution of the bands is also equivalent with that of the control. Although sample (B1) overloaded the gel, but the 564 bp band—invisible in the control due to its trace amount—is also visible in the gel.

We can conclude that the DNA samples do not degrade when being stored in coacervate. Even small amount of DNA can be recovered from the agarose gel in a good yield. It was confirmed that in the range of the markers (above 500 bp) the yield of the recovery of nucleic acids is independent of their molecular weight.

### ADVANTAGES OF THE INVENTION, INDUSTRIAL APPLICABILITY

Polymolecular complex phase-separation, such as polymolecular complex coacervation, is based on the self-assembling aggregation of surfactants, which occurs when the so-called micelles and substances forming complexes therewith interact with each other. The surfactants are capable of forming association complexes with a number of compounds, in particular with proteins and lipids. This interaction is maintained even after the surfactants are complexed, thereby a new, self-assembling entity of three basic components, such as the associated substanc(es), surfactant(s) and complexing agent(s), is formed. The lack of an overall surface charge—which can be achieved by adding an appropriate amount of the surfactant and of the complexing agent—is necessary for the coacervation of this colloidal size, newly formed entity. Thus, the novel method of the invention allows coacervation of the associated complexes, which is a significant advantage over the conventional complex coacervation.

The system of the invention is typically of self-assembling nature. Since the system seeks an energy-minimum, its components, either identical or different, are stabilized by an network of reversible, secondary interaction, i.e. they are arranged in a self-assembling way. The polymolecular complex coacervation is generally a very simple and fast process. Due to the self-assembling interactions of the components, the coacervation is performed by admixing the components and—optionally—heating the mixture. Based on the type of application, as a terminating step e.g. the equilibrium solution or the coacervate can be isolated or obtained, or the coacervate may be cooled down and dispersed.

Methods based on phase-separation offer two alternatives. On the one hand (i) they allow quick, simple and effective isolation of lipids and proteins just as substances bound to them or dissoluble in them, from the micelle rich phase, such as coacervate; as well as concentration, stabilization, separation, purification and labelling of the same. On the other hand (ii) if the equilibrium solution is utilized, the methods of the invention facilitate removing lipids and proteins—as well as substances bound to them or dissoluble in them—quickly, simply and effectively, as well as decontaminating solutions from surfactants, lipids and proteins.

Application of the polymolecular complex coacervation for nucleic acid purification is of outstanding importance. Methods hitherto used for extracting nucleic acids were based on physical (centrifugation), or physico-chemical (adsorption) or chemical (use of phenol) principles. The polymolecular complex coacervation allows colloid based separations. Since both proteins and nucleic acids form colloid solutions, the present highly appropriate colloid-based method provides gentle, quick and effective separation. Physical methods applied for the separation of nucleic acids are time-consuming, the physico-chemical methods depend on the quantity and quality of nucleic acids, while the chemical methods use toxic, aggressive or volatile materials. Quite to the contrary, the method based on polymolecular complex coacervation is quick, its scale is variable within an arbitrary range, and the reagents used are neither hazardous nor volatile. A method based on a surfactant and an amino acid derivative can be safely carried out even by high-school students. The simplicity of the method allows performing the procedure at places with no laboratory equipment (e.g. school, agricultural area, etc.). The method based on the use of surfactant is especially useful in analyzing dried biological (e.g. forensic, criminalistic) samples.

Contrary to microcapsule production based on conventional complex coacervation the method of the invention provides several advantages. The substances used are not of natural origin, thus their composition does not vary, and there is no risk of infection. The method is quicker, simpler, and less sensitive to the variation of parameters. The wall of the capsules does not necessarily require additional stabilization. Considering traditional uses of the microcapsules it becomes apparent that the method of invention can be used, among others, for microencapsulating enzymatic additives of washing powders, or for microencapsulation of plant protection agents to promote their retarded absorption or their aerial dispersion. New fields of application, such as targeting certain biomolecules are now within reach. The micro- or nanoparticles produced by the method utilizing surfactants are probably easily taken up by living cells, thereby these particles may well be suitable for in vivo targeting certain medicaments, proteins, or radioactive substances. Because of the polymolecular nature of the microparticles, it is possible to attach a molecular label or receptor molecules to their surface, providing the directed accumulation of microparticles in the living organism.

According to the present invention, in the presence of organic, preferably apolar solvents reversed micelles can be formed as well, and water soluble molecules unable to form association complexes with the micelles of the invention can be enclosed in the polar, preferably aqueous medium inside these micelles.

The novel method of the invention is also useful for the production of micro- or nanoemulsions or films. The emulsions can be used in a wide range of applications. For example, they may be used as microemulsions of injections presently causing pain, in medicaments irritating the stomach, in preparations comprising oil soluble drugs (e.g. vitamins) or flavouring substances. Since the emulsions can be appropriately dosed, and are ready-to-use, the application of emulsions in surfactants is highly advantageous. Moreover, the complexed structure of the inventive micelles, compared to anionic micelles, largely enhances the absorption oily substances, since the oily substance does not need to pass a negatively charged surface. Additionally, if the micelles filled with the oily substances are solidified at the end of the process, they can be removed readily (oil decontamination, regeneration).

The emulsions comprising complexed micelles can be applied for proteins as well. Microemulsions are preferably used in the field of polymers, adhesive production and dyestuffs.

Parallel to the development of nanotechnology, nanoemulsions and dispersions are more and more widely applied for manufacturing products ranging from catalisators to therapeutic radioactive isotopes.

Capsules or emulsions of the present invention can be produced in a biocompatible form and used for packaging, administering or targeting drugs or test compounds.

The phase separation of micellar colloid solutions according to the invention is a versatile and widely useful method. The efficiency of the method is significantly enhanced by the fact that it can be supplemented by several other well-known methods and principles and it can be combined with other procedures.

### REFERENCES

1) International Union of Pure and Applied Chemistry (IUPAC), Compendium of Chemical Terminology 2nd Edition (1997), 1972; 31, 611;
2) Xiao J X, Sivars U, Tjerneld F., Journal of chromatography. B, Biomedical sciences and applications, 2000; 743(1-2):327-38, “Phase behavior and protein partitioning in aqueous two-phase systems of cationic-anionic surfactant mixtures”;
3) Ying-fan Wang, Jeff Y. Gao, and Paul L. Dubin, Biotechnology progress, 1996; 12: 356-362 “Protein Separation via Polyelectrolyte Coacervation: Selectivity and Efficiency.”;
4) Lazko J, Popineau Y, Renard D, Legrand J., Journal of microencapsulation, 2004 February; 21(1):59-70, “Microcapsules based on glycinin-sodium dodecyl sulfate complex coacervation.”;
5) T Cosgrove, S J White, A Zarbakhsh, R K Heenan, A M Howe J Chem Soc Faraday Trans, 1996; 92:595, “Small angle neutron scattering studies of sodium dodecyl sulphate interactions with gelatin. 2. The effect of temperature and pH.”;
6) Arleth L, Bergstrom M and Skov Pedersen J., Langmuir, 2002; 18(14): 5343-5353, “Small-angle neutron scattering study of the growth behavior, flexibility and inter-micellar interactions of wormlike SDS micelles in NaBr aqueous solutions.”;
7) Griffiths, P. C., A. Y. F. Cheung, G. J. Finney, C. Farley, A. R. Pitt, A. M. Howe, S. M. King, R. K. Heenan and B. L. Bales, Langmuir, 2002; 18: 1065-1072, “Electron Paramagnetic Resonance and Small-Angle Neutron Scattering Studies of Mixed Sodium Dodecyl Sulfate and Tetradecylmalono)bis(N-methylglucamide Surfactant Micelles.”;
8) Bales, B. L., A. M. Howe, A. R. Pitt, J. A. Roe and P. C. Griffiths, J. Phys. Chem., 2000; 104: 264-270, “A Spin-Probe Study of the Modification of the Hydration of SDS Micelles by Insertion of Sugar-Based Nonionic Surfactant.”;
9) Ranganathan, R., M. Peric and B. L. Bales, J. Phys. Chem., 1998; 102: 8436-39, “Time-resolved Fluorescence Quenching Measurements of the Aggregation Numbers of Normal Sodium Alkyl Sulfate Micelles well above the Critical Micelle Concentrations.”; 10) Wallace, D. M., Methods Enzymol., 1987; 152, 41-48. “Precipitation of nucleic acids”
   Patents and Patent Applications:
   1) U.S. Pat. No. 2,800,457,
   2) WO 0005446,
   3) U.S. Pat. No. 6,488,870,
   4) U.S. Pat. No. 4,801,691,
   5) WO 9600228

1. A condensed phase composition comprising a micellar colloid system wherein the micelles are charge neutralized and comprise at least the following as structural components:
a) an anionic surfactant wherein said anionic surfactant is capable of forming hydrogen bonds and carries a negatively charged hydrogen acceptor group and
b) a small molecular cationic complexing agent soluble in a polar, protic solvent, wherein the complexing agent is a hydrogen donor compound wherein said cationic complexing agent along with ionic interaction, can form complexes with the anionic surfactant via at least hydrogen bond as a secondary interaction.

2. The composition of claim 1, wherein in the said anionic surfactant the hydrogen acceptor group is a phosphate, carbonate, sulphate or sulphonate group, and the hydrophobic moiety of the anionic surfactant is a substituted or unsubstituted alkyl, alkenyl, alkinyl, aryl or aralkyl group; and the cationic complexing agent is a compound comprising ammonium cation or guanidinium cation.

3. The composition of claim 2, wherein the cationic complexing agent is a compound comprising a cation of general formula I:
R,R′,R″NH⁺, (I)
wherein,
R is hydroxyl or substituted or unsubstituted alkoxy, alkyl, alkenyl, alkinyl, aryloxy or
aryl group, or substituted or unsubstituted amino group,
R′ and R″ are H or substituted or unsubstituted alkyl, alkenyl, alkinyl or aryl group, and R′ and R″ are identical or different, or
a compound comprising the cation of general formula II:
R—NH—C⁺(NH2)-NH—R′, (II)
wherein,
R′ and R″ are H or substituted or unsubstituted alkyl, alkenyl, alkinyl or aryl group, and R′ and R″ are identical or different.

4. The composition of claim 3, wherein the compound comprising the cation of any of the general formulae I or II comprises alkoxy, aryloxy, alkyl, alkenyl, alkinyl or aryl group that is substituted with one or more amino or imino group or with a group comprising one or more amino or imino group,
preferably,
one or more, preferably one of R, R′ and R″ in the general formula I, or one or more, preferably one of R and R″ in the general formula II is a group of general formula III:
—[(CH₂)ₓ—NH—]ₙH, (III)
wherein,
n is any integer from 1 to 10, preferably n is 1, 2, 3, 4, 5, or 6,
x is any integer from 1 to 8, preferably x is 1, 2, 3, 4, 5, or 6, and x can be identical or different in the repeating units.

5. The composition of claim 1, wherein the cationic complexing agent is selected from the following:
detectable molecules,
molecules carrying a functional group capable of forming a covalent bond; preferably a group capable of binding a conjugate and/or a group capable of forming a crosslink,
molecules having a conjugated moiety suitable for being recognized biologically (e.g. an epitope) or a conjugated moiety capable of biological recognition (e.g. a receptor),
molecules comprising more than one complexing group, thereby preferably capable of forming complexes with more than one micelles simultaneously.

6. The composition according to claim 1, wherein said composition comprises a single homogeneous phase which is either in solid form and comprises micelle-crystals, or is a coacervate.

7. The composition according to claim 1, wherein the micelles of said composition comprise further substances, preferably proteins, nucleic acids or apolar substances, such as lipids, colouring agents or dyes, pharmacologically active molecules, cosmetics or synthetic polymers enclosed in the micelles or bound to the micelles for example by forming complexes with the micelles.

8. The composition according to claim 1 wherein the small molecular cationic complexing agent is capable of effecting coacervation in a solution comprising a polar, protic solvent and an anionic surfactant in a concentration above Critical Micelle Concentration at a pH level ensuring that the anionic surfactant is in a charged form and,
if added in an amount sufficient to neutralize the charges of the anionic surfactant,
preferably at a temperature above the Kraft point.

9. The composition of claim 8 wherein the small molecular cationic complexing agent is soluble in water and the hydrating water molecules are excluded by the small molecular cationic complexing agent.

10. A micro- or nanocapsule comprising the composition of claim 1, or a nanocapsule comprising a single micelle of the composition of claim 1.

11. A method for the preparation of a colloid system comprising micelles formed by an anionic surfactant, and for phase-separation thereof to a micelle rich phase and to an equilibrium solution, preferably for coacervation, characterized in that
as a micelle rich phase, a self-assembling colloid system is prepared at least from the following:
a) a polar, protic solvent or solution containing it,
b) at least one type of anionic surfactant capable of forming micelles when exposed the solvent or the solution, wherein said anionic surfactant carries a negatively charged hydrogen acceptor group and is capable of forming hydrogen bonds,
c) at least one type of small molecular, cationic complexing agent with hydrogen donor property, which is soluble in the solvent and which, along with ionic interaction, is capable of forming complexes with the surfactant via at least hydrogen bond,
wherein
the anionic surfactant is used in a concentration above Critical Micelle Concentration at a pH level ensuring that the anionic surfactant is in a charged form,
the cationic complexing agent is added to a concentration suitable for neutralizing the charges in the micelles,
after addition of the surfactant and the cationic complexing agent the system is optionally homogenised, and
phase-separation, preferably coacervation, of the colloid system is initiated by providing an appropriate temperature preferably at a temperature above the Kraft point and, if desired, by adjusting or changing the pH, ionic strength and/or dielectric constant.

12. The method of claim 11, wherein
the polar, protic solvent is water and the hydrating water molecules are excluded by the small molecular cationic complexing agent.

13. The method of claim 12, wherein the hydrophobic moiety of the anionic surfactant is a substituted or unsubstituted alkyl, alkenyl, alkinyl, aryl or aralkyl group, while the hydrophilic moiety is a sulphate or sulphonate group or a substance comprising these groups and the cationic complexing agent is a substance comprising ammonium cation or guainidium cation.

14. The method of claim 13, wherein the cationic complexing agent is a compound comprising a cation of general formula I:
R,R′,R″NH⁺, (I)
wherein,
R is hydroxyl or substituted or unsubstituted alkoxy, alkyl, alkenyl, alkinyl, aryloxy or
aryl group, or substituted or unsubstituted amino group,
R′ and R″ are H or substituted or unsubstituted alkyl, alkenyl, alkinyl or aryl group, and R′ and R″ are identical or different, or
a compound comprising the cation of general formula II:
R—NH—C⁺(NH2)-NH—R′, (II)
wherein,
R′ and R″ are H or substituted or unsubstituted alkyl, alkenyl, alkinyl or aryl group, and R′ and R″ are identical or different.

15. The method of claim 11, wherein contaminants incorporated in or bound to the micelles composing the self assembling colloid system, e.g. a contaminant substance forming a complex with the micelles, are eliminated from an aqueous solution, said method comprising the steps of
producing micelles, by addition of the anionic surfactant, in the aqueous solution, said micelles comprising the contaminants or forming complex with the contaminants,
initiating phase-separation by using cationic complexing agent, and
separating the micelle rich phase, and
repeating the procedure until a sample sufficiently free of contaminants is obtained.

16. The method of claim 15, wherein an apolar contaminant is removed from aqueous solution.

17. The method of claim 15, wherein the anionic surfactant is a sulphate and/or sulphonate comprising alkyl and/or aryl group, preferably SDS or SDBS, and the formation of emulsion is avoided by appropriate selection of the anionic surfactant and the cationic complexing agent.

18. The method of claim 11, wherein a substance incorporated in or bound to the micelles composing the self-assembling colloid system, e.g. a substance forming a complex with micelles, is isolated from an aqueous solution, said method comprising the steps of:
producing micelles, by addition of the anionic surfactant, in the aqueous solution, said micelles comprising the target substance or binding the target substance,
initiating phase-separation by using the cationic complexing agent, and
separating the micelle rich phase, and
recovering the target substance, incorporated in or bound to the micelles, from the micelles.

19. The method of claim 18, wherein the target substance is a protein or wherein the target substance is a nucleic acid.

20. The method of claim 11, wherein at least two substances incorporated in or bound to the micelles composing the self-assembling colloid system—e.g. substances forming a complex with the micelles—are isolated from an aqueous solution, said method comprising the steps of:
adding an anionic surfactant to an aqueous solution comprising the proteins in an amount less than the amount necessary to reach saturation of the proteins,
adding the cationic complexing agent to the solution in excess relative to the amount of the anionic surfactant, thereby initiating coacervation,
by adjusting the pH of the solution, initiating a partition of the proteins between the coacervate and the equilibrium solution, wherein said partition is based on differences in the isoelectric points of the proteins,
isolating the desired protein by separation of the two phases, on the basis of phase-partition, optionally by chromatography.

21. The method of claim 11, wherein, following the phase-separation, the micelle rich phase comprising the target substance or micelles bound to the target substance is converted into micro- or nanodispersion or to micro- or nanocapsules.

22. The method of claim 21, wherein the phase-separation is coacervation, and the coacervate obtained by coacervation is emulsified in a suitable solvent, then microcapsules or nanocapsules are formed from the emulsion, or wherein a system that coacervates above the temperature of the desired application is chosen, a coacervate is produced by using the said chosen system, then microcrystalline dispersion or capsules are obtained by cooling the coacervate.

23. A condensed phase composition comprising a micellar colloid system wherein the micelles are charge neutralized and comprise at least the following as structural components:
a) an anionic surfactant wherein said anionic surfactant is capable of forming hydrogen bonds and carries a negatively charged hydrogen acceptor group and
b) a small molecular cationic complexing agent soluble in a polar, protic solvent, wherein the complexing agent is a hydrogen donor compound wherein said cationic complexing agent along with ionic interaction, can form complexes with the anionic surfactant via at least hydrogen bond as a secondary interaction,
wherein said composition is obtained by the method according to claim 12.
